(19) United States
(12) Patent Application Publication
(10) Pub. No.: US 2011/0229572 A1
(43) Pub. Date: Sep. 22, 2011

(54) DELIVERY OF DRUG COMBINATIONS

(75) Inventors: Andrew Lennard Lewis, Farnham, Surrey (GB); Richard Edward John Forster, Singapore (SG); Mroa Voctoria Gonzalez-Fajardo, Madrid (ES); Yiqing Tang, Surrey (GB); Andrew Willam LLoyd, Brighton (GB); Gary John Phillips, Brighton (GB)

(73) Assignee: BIOCOMPATIBLES UK Limited, Farnham, Surrey (GB)

(21) Appl. No.: 12/672,031

(22) PCT Filed: Aug. 18, 2008
(86) PCT No.: PCT/GB08/50722

§ 371 Date: Jul. 28, 2010

(30) Foreign Application Priority Data
Aug. 16, 2007 (GB) 0716003.9
Nov. 20, 2007 (EP) 07121158.5

(51) Int. Cl. A61K 9/00; A61K 31/704; A61K 31/4745; A61K 31/436; A61K 31/192; A61P 35/00; A61K 9/14

(52) U.S. Cl. 424/487; 424/484; 514/34; 514/283; 514/291; 424/486; 514/570; 427/2.14

(57) ABSTRACT

A composition comprising microspheres of a polymer matrix, having two different pharmaceutical actives having complementary, usually synergistic, activity in killing cells. The compositions have particular utility for treating tumours. Useful combinations are doxorubicin with rapamycin, irinotecan with ibuprofen, ibuprofen with doxorubicin and irinotecan with doxorubicin. The polymer matrix is preferably a crosslinked polyvinyl alcohol. The drugs may be included in the same microsphere, or microspheres each with an individual pharmaceutical agent may be mixed together. The microspheres are preferably used in chemoembolisation of tumours.

The present invention relates to drug delivery articles, usually microspheres, formed of a polymer matrix and at least two drugs, the drugs being releasable from the polymer matrix. The drugs are for treatment of cancer, especially solid tumours.

Combination therapy has been the basis for most success stories in cancer treatment, which is understandable when components of a combination have a favourable pharmacological interaction (same target, but different large-organ toxicities). Combination therapy with multiple drugs is a clinically common procedure in tumour treatment. The most common therapeutic regimens for ovarian, prostate or uterine cancer are based in combination of two antitumor drugs. This type of therapy could offer the advantage of increasing the tumoricidal efficacy and reducing the side effects caused by the high-dose of a single drug. Irinotecan is indicated in combination with 5-FU for the treatment of patients with advanced colorectal cancer and it has been used with success in combination with other agents such as epirubicin, mitomycin or capecitabine to treat breast cancer (Becerra 2004, Oncology, 18, 46-48), ovarian cancer (Nishino et al. 2005, Gynecol Oncol, 97, 893-897) or gastric cancer (Baek et al. 2006, Br J Cancer, 25, 312-320) respectively.

A more recent foundation for the success of some cancer treatments involves a mechanistic basis, under which a combination of drugs is used to affect different molecular targets to circumvent resistance. A barrier to major advancements in combination therapy, however, has been a lack of understanding about the intersection of critical signalling pathways. Synergy might be induced through the effect of drugs on the same as well as parallel pathways. Because the number of drug combinations is limitless, a strategy for determining the most promising combinations and prioritizing their evaluation is crucial.

Mathematical modelling and computer simulations are used to calculate the expected effect of different drug combinations in an effort to better understand how chemotherapeutic combinations might be used clinically. In vivo, favourable synergistic drug combinations may result in increased efficacy, decreased dosage, reduced toxicity, and minimized development of resistance. The Median Effect Equation (Chou J. Biol. Chem. 1977, 252(18), 6438-6442) and Combination Index Equation (Chou-Talalay T.I.P.S. November 1983 450-454) are two such treatments in which the isobologram curves generated represent additive, synergistic, or antagonistic interactions caused by different drug combinations. The concept behind and applications of surface response models encompasses the joint effect of two active agents which can be viewed as a three-dimensional response surface. While these models for assessing joint action are the most comprehensive, they are also highly complex and difficult to create. Indeed, it is recognised that the true link between empirical synergy and the underlying biochemical, molecular, and physiological mechanisms is very difficult to discover.

A fluorescence-based, high-throughput system, called digital image microscopy-based cytotoxicity assay (DIMSCAN), exists for in vitro testing of cytostatic or cytotoxic drug combinations. There are five principles for this type of testing: (a) the assay system should have a wide dynamic range, ideally 3 to 4 logs of cell kill; (b) the cell line panel should employ multiple cell lines, including drug-resistant lines; (c) major mechanisms of resistance should be identified and used to structure the cell line panel; (d) exposure to drugs should be at clinically achievable levels and schedules; and (e) because hypoxia may antagonize drug action, it is essential to test under hypoxic conditions. The best approach for selecting drug combinations for evaluation is the one based on molecular targets; studies should first be performed in vitro and then confirmed with in vivo models before clinically testing the drug combination. Drug antagonism is also frequently used to exclude drug combinations.

There are known synergistic interactions between novel signal transduction modulators and established cytotoxic agents, cell cycle inhibitors, and differentiation inducers. While the first type of drug combination involves toxic concentrations of the cytotoxic agent to produce modest potentiation in vitro, the second and third types induce a high degree of synergism at concentrations of both agents that are nontoxic (ineffective) when given individually. Drug combinations between cell cycle inhibitors or these agents and differentiation inducers have also been shown to produce significant synergistic activity in vitro. The number of signal transduction modulator combinations associated with synergistic interactions of cell death is virtually limitless. Whether these combinations are therapeutically superior to those between conventional cytotoxic agents or those between conventional and novel agents and whether they represent a new paradigm for combination chemotherapy remains to be determined.

Celator Technologies, Inc., in Canada, have reported on commercial solutions for bringing effective drug combinations to the clinic. Celator is working on the identification of critical ratios at which drug combinations act synergistically to kill tumour cells, with the objective of fixing those synergistic ratios in a liposome-based delivery vehicle (CombiPlex) designed to target tumors following i.v. injection. Here, whilst the in vitro synergistic activity depends on specific drug ratios, the in vivo activity depends on maintaining these synergistic ratios. The drugs are incorporated jointly into the liposomes.

Their systems have been described in patent publications, such as WO2003028696, WO2004087115, WO2004087105, WO2004093795, WO2006055903, and in Tardi, P. G. et al, (2007) Biochim. Biophys. Acta 1768, 678-587.

Microspheres offer a particularly versatile platform for delivery of drug combinations. Sharma et al have investigated microspheres for inhalation of drug combinations to treat tuberculosis (Sharma R, at al, 2001, Pharm Research, 18, 1405-1410). Drug therapy of tuberculosis (TB) requires long-term oral administration of multiple drugs for curing as well as preventing and/or combating multi-drug resistance. Persistent, high blood levels of antitubercular drugs resulting from prolonged oral administration of anti-TB drugs may be neither necessary nor sufficient to kill mycobacteria residing in macrophages (Mφ). Inhalable biodegradable microparticles containing two of the first-line anti-TB drugs, isoniazid (H), and rifampicin (R), were prepared and tested for (i) phagocytosis by mouse Mφ (ii) administration as a dry powder inhalation to rats, and (iii) targeting alveolar Mφ with high drug doses when administered to rats. Results showed that such microspheres with multiple drugs offer promise for dose and dose-frequency reduction, toxicity alleviation and targeting of Mφ-resident persistent mycobacteria.

In another study (Gupte A & Ciftci K, 2004, Int. J. Pharm,. 276 (1-2):93-106.), the combination (Paclitaxel+5-FU microspheres) was compared with a single drug chemotherapy (Paclitaxel and 5-FU microspheres) against metastatic breast cancer cell line (MDA-MB 435 S). The physicochemical characteristics of the microspheres (i.e. encapsulation efficiency, particle size distribution, in vitro release, thermal characteristics) were studied. The results demonstrated that the encapsulation efficiency of Paclitaxel was high (90%) when the drug was encapsulated in poly(lactic-co-glycolic acid) (PLGA) microparticles with or without 5-fluorouracil (5-FU). However, the encapsulation efficiency of 5-FU was low (19%) and increased to 30% when the drug was encapsulated with Paclitaxel. The mean particle size of microspheres was 2.5 μm and were spherical in shape. The in vitro release of both 5-FU and Paclitaxel from the microspheres was relatively fast initially followed by a slower and more controlled release. The cytotoxic activity of Paclitaxel microspheres was far greater compared to either the microspheres containing 5-FU+Paclitaxel or 5-FU alone. Overall results demonstrated that incorporation of Paclitaxel or 5-FU in microspheres enhances the cytotoxicity in more controlled manner compared to that of free drugs and also that careful consideration should be made when combining drugs acting in different phases of cell cycle.

Others have investigated drug combinations in microspheres to target restenosis (Chandy et al, 2001, Development of Poly(Lactic Acid)/Chitosan Co-Matrix Microspheres Controlled Release of Taxol-Heparin for Preventing Restenosis, Drug delivery, 8, 77-86). Smooth muscle cell proliferation plays a major role in the genesis of restenosis after angioplasty or vascular injury. Controlled release of appropriate drugs alone and in combinations is one approach for treating coronary obstructions, balloon angioplasty, restenosis associated with thrombosis, and calcification. Chady et al, demonstrated the possibility of encapsulating taxol-loaded polylactic acid (PLA) microspheres within heparin-chitosan spheres to develop a prolonged release co-matrix form. The in vitro release profile of taxol and heparin from this co-matrix system was monitored in phosphate buffered saline pH 7.4, using an ultraviolet spectrophotometer. The amount of taxol/heparin release was initially much higher, followed by a constant slow release profile for a prolonged period. The initial burst release of taxol (15.8%) and heparin (32.7%) from the co-matrix was modified with polyethylene glycol coatings (13.5% and 25.4%, respectively, for 24 hr). From scanning electron microscopy studies, it appears that these drugs diffuse out slowly to the dissolution medium through the micropores of the co-matrix. However, the surface micropores were modified with polyethylene glycol (PEG) coatings for a constant slow release profile. They concluded that this PEG-coated PLA/chitosan co-matrix may target drug combinations having synergistic effects for prolonged periods to treat restenosis.

Cardiovascular stents coated with a combination of drugs have been described for reducing restenosis rate. Restenosis is a hyper proliferative disease. For instance EP-A-551162 discloses a combination of rapamycin and mycophenolic acid, an anti-proliferative agent, but does not give details of how a stent is to be impregnated with the actives. In EP-A-0568130 rapamycin and heparin are administered to prevent smooth muscle hyperplasia, e.g. by delivery from an impregnated stent. No details are given of impregnation methods.

WO-A-2003022807 describes administration of a rapamycin analogue from a stent coated with a polymer. The drug is loaded by dipping the polymer coated stent into a solution of the drug and drying. In addition a further drug such as an anti-miotic, anti-proliferative or anti-inflammatory agent may be administered from the stent. Polymers include among many others polyvinyl alcohol, hyaluronic acid and copolymers of phosphorylcholine methacrylate (MPC), nonionic comonomers and crosslinking monomers, and these last mentioned copolymers are exemplified in in vitro tests, with a rapamycin analogue alone. In WO2004/022124 a rapamycin analogue and a dexamethasone are jointly loaded into a crosslinked MPC copolymer with non-ionic comonomer on a stent. The polymer used in these specifications has a water-swellability of about 50%.

5-Fluorouracil (5-FU) in combination with leucovorin (LV) is nowadays the standard treatment in colon cancer. Lamprecht et al (Eur J Pharm and Biopharm, 2005, 59, 367-371) used Eudragit P-4135F or Eudragit RS100 separately to prepare microspheres by an oil/oil emulsification process trapping 5-FU and LV simultaneously. Scanning electron microscopy permitted a structural analysis, process parameters were analyzed and drug loading and release profiles were recorded. Particle size varied between 123 (RS100) and 146 μm (P-4135F). Generally, higher encapsulation rates were found with RS100 (5-FU, 60.3±9.7%; LV, 81.4±8.6%) compared to P-4135F (5-FU, 48.3±2.0%; LV, 55.4±2.7%). These Eudragit polymers are acrylic copolymers including quaternary ammonium group containing alkyl methacrylate monomers. They are not crosslinked. Microparticles made from Eudragit RS100 released the incorporated drug combination within 8 h. P-4135F was found to maintain the undesired 5-FU release at pH 6.8 lower than 25% within 4 h while at pH 7.4, a nearly immediate release (within 15 min) was observed. Although the release was similar at pH 7.4, at pH 6.8 LV showed a distinct initial drug loss of about 60% and a complete release within 2 h. SEM analyses revealed a substantial presence of LV crystals on the particle surface provoking a distinct burst effect of LV. These observations were concluded to be related to the high lipophilicity of P-4135F provoking a separation between P-4135F and LV during the preparation process.

Liu et al. (J. Pharm. Pharmacol., 55, 1063-1073, 2003) and in WO9850018 describe the use of biodegradable sulfopropyl dextran microspheres for intratumoural injection. In the method microspheres containing doxorubicin are co-administered with microspheres containing verapamil. Following intratumoural injection in mice of doxorubicin-loaded microspheres, alone or in combination with verapamil-loaded microspheres, the tumour diameter was measured serially as an indication of therapeutic effect, while the weight, appearance, and behaviour of the mice were monitored as an indication of general toxicity. Intratumoural injections of doxorubicin-loaded microspheres were tolerated much better than systemic administration of equivalent drug concentrations. There was a modest (up to 34%) delay of tumour growth compared with groups receiving no treatment or blank microspheres. Co-injection of verapamil microspheres with doxorubicin microspheres produced a moderate increase in toxicity but no further delay in tumour growth. Controlled-release microsphere formulations of anticancer agents administered intratumourally were described as an efficient way to deliver high drug doses to the tumour with little systemic toxicity. The sulpho-propylated dextran microspheres used had been supplied as ion-exchange media (Sephadex SP25). They are biodegradable. They are swellable in distilled water to give a volume ratio of microspheres in the swollen state to dry state of about 12. The microspheres have an ion-exchange capacity of about 2.5 meq/g at pH 7.

Liu et al. in (2000) J. Pharm. Sci: 8-9 (6) 807-817 have also described ways of modifying the surface of the sulpho-propylated microspheres with hydrophobic moieties or with cationic polymers. Cationic polymer modification reduces the swellability ratio and ion-exchange capacity. The surface conjugation of hydrophobic groups affects the rate of swelling in water but does not reduce the extent of swelling at equilibrium verapamil loaded at a more rapid rate into the polymer treated microspheres.

Doxorubicin eluting beads have been shown to be effective in the treatment of HCC by chemoembolisation (Varela et al., 2007, J Hepatology, 46, 474-81). Microspheres for delivering doxorubicin to solid tumours by chemoembolisation are described in WO2004071495. The microspheres are formed of a matrix of water-swellable anionically charged crosslinked polyvinyl alcohol and have diameters when swollen in water in the range 40 to 1500 μm.

Ibuprofen eluting beads have demonstrated anti-inflammatory effects post embolisation in animal models (Borovac T, et al, 2006, J Control Release, 115, 266-74). Microspheres for use in the treatment of uterine fibroids loaded with ibuprofen are described in WO2004073688. Improved loading methods are described in WO2007085615.

Ibuprofen is an non-steroidal anti-inflammatory drug (NSAID), which acts by inhibition of the enzymes cyclooxygenase I and II. Many authors have studied the use of NSAIDs for treatment and prevention of cancer risk (Sawaoka et al. 1998, J Clin Gastroenterol, 27, 47-52; Shen et al. 1998, Cancer Research, 58, 362-366). COX-2 selective NSAIDs have also demonstrated a reduction in cancer cell invasion and liver metastases (Yamauchi et al. 2003, Anticancer Res, 23, 245-249; Yao et al. 2004, Br J Cancer, 90, 712-719) and inhibition of COX-2 resulted in reduced tumour size and increased survival (Cui et al. 2005, Clin Cancer Res, 11, 8213-8221; Murata et al. 2005, Dig Dis Sci, 50, 70-75). Ibuprofen has been investigated in the treatment and prevention of a number of tumours. In WO2006013376 we describe the use of microspheres loaded with various COX inhibitors for embolisation showing elution rates and localisation which lead to an expectation that an effect would be shown on tumourigenesis.

In WO2007090897, we describe methods of loading water-swellable polymers, such as microspheres, with drugs having a very low solubility, but which are soluble in an organic solvent, in which a solvent based solution of the drug is contacted with substantially dry polymer matrix, so that swelling of the matrix in the drug solution takes place, followed by precipitation of the drug in the matrix of the polymer by contacting with a precipitant, usually water. The method is suitable for loading paclitaxel, dexamethasone or rapamycin. The polymer matrix is a crosslinked polyvinyl alcohol based material, usually having pendent anionic groups, such as sulphonate groups.

In WO2006027567 we describe microspheres, for chemoembolisation of solid tumours comprising a water-swellable polymer which is anionically charged at pH 7 and, electrostatically associated with the polymer in releasable form, a cationically charged camptothecin compound, such as irinotecan. We have shown that chemoembolisation using these beads for treatment of liver metastases from colorectal cancer leads to a reduction in tumour volume.

According to a first aspect of the present invention there is provided a composition comprising a population of microspheres each of which has a polymer matrix and, jointly incorporated into the matrix, a first and a second pharmaceutically active compound, wherein the polymer is a water-insoluble water-swellable polymer which is anionically charged, the first active is cationically charged, one of the actives is a cytotoxic compound and the other active has activity complementary to the cytotoxic compound in tumour treatment.

In this aspect of the invention, the first cationically charged active may be the cytotoxic compound. Alternatively the second active may be the cytotoxic compound, while the first cationic active has activity complementary to the cytotoxic compound in tumour treatment. The second active may be cationically charged, or may be neutral. In some embodiments it may be possible to utilise anionically charged actives, although these are less preferred. Ionically charged actives tend to be water-soluble at a relatively high level, for instance at a level of at least 50 g/l, preferably at least 100 g/l. The second active, when a neutral compound, may be less water-soluble, for instance having a low solubility in water, less than 10 g/l, at room temperature. We have found that for the combination of cationic and nonionic drugs, there is little or no interference with the loading or release rate of each active by the other. This result is surprising and allows the ready determination of suitable dosages and ratios of composition which are optimised for efficacy. The composition will contain particles of a single type which avoids problems of separation causing dosage uncertainties.

The second active has an activity which is complementary to the cytotoxic compound in tumour treatment. The compound may itself be efficacious in treatment of tumours, for instance be capable of preventing growth or reducing the size of a tumour, for instance when delivered locally, or systemically. The active may alternatively be a compound which sensitises the tumour to activity by the first active. In general the combination of compounds may be identified as one which has a combination index based on the Chou-Talalay index (1983, op. cit), of less than 0.90, more preferably less than 0.5, for instance less than 0.3, even as low as 0.1 or below.

Values for the combination index are determined, conveniently, using the software package Calcusyn (Biosoft), which is a dose effect analysis programme utilising the general equation for dose effect relationships derived by Chou:

fₐ/fᵤ=(D/Dₘ)^{m} Eq. 1.

where
D: the dose of drug
Dₘ: the median-effect dose signifying the potency. It is determined from the x-intercept of the median-effect plot.
fₐ: the fraction affected by the dose
fᵤ: the fraction unaffected, fu=1-fₐ
m: an exponent signifying the sigmoidicity (shape) of the dose effect curve. It is determined by the slope of the median effect plot.

The alternative forms of the median-effect equation are:

fₐ=1/[1+(Dₘ/D)^{m}] Eq. 2. D

=Dₘ[fₐ/(1−fₐ)]^{1/m} Eq. 3.

From Eq. 2, if Dₘ and m are known, the effect (fₐ) can be determined for any dose (D).

From Eq. 3, if Dm and m are known, the dose (D) or (D_{X}) can be determined for any effect (fₐ). Thus, Dₘ and m parameters representing the potency and shape, respectively, determine the entire dose-effect curve.

The median-effect plot is a plot of x=log(D) vs y=log (fₐ/fᵤ) introduced by Chou in 1976 (Chou J. Th. Biol. 59, 253-276). It is based on the logarithmic form of Chou's median effect equation [Eq. 1]:

log(fₐ/fᵤ)=m log(D)−m log(Dₘ) Eq. 4

Eq. 4 has the form of a straight line, y=mx+b.

Thus, the slope yields m value and the x-intercept yields log(Dₘ) value, and thus, the Dₘ value. The Dₘ can also be determined by: Dₘ=10^{-(y-intercept)/m)}

Note that fₐ/fᵤ=fₐ/(1−fₐ)=(fᵤ)⁻¹−1=[(fₐ)⁻¹−1]⁻¹. The goodness of fit for the data to the median-effect equation is represented by the linear correlation coefficient r of the median-effect plot. Usually, the experimental data from enzyme or receptor systems have r>0.96, from tissue culture r>0.90; and from animal systems, r>0.85.

The combination index (CI) equation is based on the multiple drug-effect equation of Chou-Talalay derived from enzyme kinetic models. An equation determines only the additive effect rather than synergism or antagonism. However, we define synergism as a more than expected additive effect, and antagonism as a less than expected additive effect. Chou and Talalay in 1983 (op. cit) proposed the designation of CI=1 as the additive effect, thus from the multiple drug effect equation of two drugs, we obtain:

Classical: for mutually exclusive drugs that have the same or similar modes of action:

CI=(D)₁/(Dₓ)₁+(D)₂/(Dₓ)₂ Eq. 5

Conservative: for mutually non-exclusive drugs that have totally independent modes of action:

CI=(D)₁/(Dₓ)₁+(D)₂/(Dₓ)₂(D)₁(D)₂/(Dₓ)₁(Dₓ)₂ Eq. 6

Eq. 5 or Eq. 6 dictates that drug 1, (D)₁, and drug 2, (D)₂, (in the numerators) in combination inhibit x % in the actual experiment. Thus, the experimentally observed x % inhibition may not be around number but most frequently has a decimal fraction. (Dₓ)₁ and (Dₓ)₂ (in the denominators) of Eq. 5 or 6 are the doses of drug 1 and drug 2 alone, respectively, inhibiting x %. Dₓ can be readily calculated from Eq. 3.

It is worth noting that each drug alone should have a dose-effect relationship. Both potency (Dₘ) and shape (m) parameters are essential for determining synergism or antagonism. If one of the two drugs has no effect by itself, then synergism/antagonism cannot be determined. Instead, potentiation (augmentation, enhancement) or inhibition (suppression) can be determined.

Calcusyn requires the dose-effect relationship for each drug and then for combinations. Although the experimental data from the combined study is preferably collected using a constant ratio, combination indices may nevertheless be determined from non-constant ratios of the two components.

The combination index may be determined for any in vitro or in vivo test method. In the present invention, it is most convenient for the combination index to be determined in vitro using a cell culture based test, for instance on a cell line of cells representing a useful in vitro test for determining in vivo efficacy. For instance the test may determine the cytotoxicity of the individual drugs and then of the combination, and these data used to calculate the combination index. In the test, the drugs may be contacted with the cells in solution, or in some other form. The effect which is to be measured is cytotoxicity, such as reduction in 50% viable cell count compared to control after a given period such as 24 or 48 hours. It is not necessary for the drugs to be incorporated into a polymer matrix, for instance in the form of microspheres, to carry out the test. However it is preferred that the combination index is determined to be less than 1 when the drugs are delivered from a polymer matrix, for instance in the form of microspheres as defined in the present claims. In this case, the combination of drugs tested may involve separate particles, each loaded with one of the drugs or each particle may be jointly loaded with both the drugs. A suitable test is described in Examples 6 and 7 below. Where separate particles are each loaded with one of the drugs it should be checked that, for the joint-loaded particles the elution rate of each drug is not significantly affected by the other if it is to be assumed that the determined CI value will apply to the joint-loaded product. In this aspect of the invention the combination of pharmaceutically active compounds is one which has a CI of less than 1.0 in an in vitro cytotoxicity test on a tumour cell line at a ratio within a multiple of 5 (either way) of the ratio in the composition. Where the composition is for treatment of a hepatocellular carcinoma the cell line used to determine the CI is preferably a hepatocellular carcinoma cell line. Where the composition is for treating pancreatic carcinoma, the cell line is preferably a pancreatic carcinoma cell line. The CI should preferably be the defined low value when the active compounds are tested at a ratio within a multiple of 2 either way of the ratio in the composition.

A cytotoxic agent is one that is directly toxic to cells, preventing their reproduction or growth. Suitable cytotoxic agents are, for instance, anthracycline compounds such as doxorubicin and other compounds disclosed in WO04071495, camptothecin derivatives as described in WO2006027567, taxanes, platinum-based neoplastic anti-metabolites such as 5-FU, mercaptopurine, capecitabine other cytotoxic antibiotics such as actinomycin D and vinca alkaloids, including vinblastine, vincristine, vindesine and vinorelbine. Examples of cytarabine, gemcitabine, cyclophosphamide, fludaribine, camptothecin compounds such as topotecan and irinotecan, clorambucil, busulfan, mitoxantrone, retinoids, anagrelide etc.

The active which provides complementary activity may be a cytostatic compound. Cytostatic compounds may be defined as compounds that inhibit or suppress cellular growth or reproduction. The cytotoxic and cytostatic effects of drugs may be determined by the person skilled in the art, using suitable tests, such as based on cell lines.

Suitable cytostatic compounds are rapamycin and analogs.

Suitable cationically charged compounds are: doxorubicin and other anthracycline compounds having an amine group as disclosed in WO04071495 or mitoxantrone, cationic camptothecin derivatives, such as irinotecan, as described in WO2006027567, the contents of which are incorporated herein by reference, or topotecan, or verapamil.

One class of cytotoxic or cytostatic compounds which may be used comprises rapamycin and rapamycin analogues, which target mTOR. Such compounds include sirolimus, temsirolimus, everolimus, biolimus, ABT-578 and AP23573. Any of the compounds encompassed within the scope of rapamycin analogues described in WO-A-2003022807, the contents of which are incorporated herein by reference, may be used as the rapamycin analogue.

Where the cytotoxic active is rapamycin or a rapamycin derivative/analogue, the other active is in one preferred embodiment, doxorubicin or another anthracycline compound having an amine group as described in WO04071495.

The combination of anticancer drug rapamycin and doxorubicin has been demonstrated a complete remission of a mouse model of B-cell lymphoma through the reverse of chemoresistance in lymphomas by rapamycin sensitisation, and induction of apoptosis by doxorubicin (Wendel, H-G, et al, 2004, Nature 428, 332-337). In the treatment of leukemias, rapamycin has shown the enhancement of doxorubicin-induced apoptosis (Avellino, R. et al, 2005, Blood 106(4), 1400-1406). Combinations of rapamycin or an analogue with an anthracycline compound having at least one amine group show a low combination index, and are highly effective in the present invention.

In another embodiment of this aspect of the present invention the cytotoxic compound is rapamycin or an analogue and the cationic active is a camptothecin derivative having a cationic substituent as disclosed in WO2006027567 or in EP-A-0321122. Preferably the active is as defined in claim 13. Preferably A is COO. Preferably AR is substituted in the 10 position.

In another aspect of the present invention (the further or second, aspect hereinafter) a pharmaceutical product comprises a first population of microspheres comprising a first polymer matrix and a first therapeutically active agent and a second population of microspheres comprising a second polymer matrix and a second therapeutically active agent, wherein said first and/or second polymer matrix(es) is (are) crosslinked water-swellable polymer and the first therapeutically agent and second therapeutically active agent are incorporated into the microspheres and the populations are present in a unit dose of product in amounts such that the Combination Index, as hereinbefore defined, is less than 0.1.

In this aspect of the invention the above combinations of cytotoxic and cytostatic agents described in connection with the first aspect of the invention and which have the defined Combination Index may be used. The composition is used in a method of treatment of a tumour, preferably a solid tumour. For this aspect the CI should be determined for the system with the actives loaded in their respective microspheres, i.e. of the composition of this aspect of the invention, i.e. jointly or separately loaded as the case may be.

In this aspect of the invention, the drugs may each be incorporated into separate population of microsphere. Preferably, however, the populations are the same, that is the two drugs are jointly loaded onto the same microspheres. Where the cytotoxic compound is rapamycin or an analogue, the active having complementary activity may alternatively be selected from cisplatin, or other platinum based anti-cancer drug, gemcitabine, tamoxifen or another anti-estrogen, interferon alpha, epithelial growth factor receptor inhibitor, vascular endothelial growth factor inhibitor, inhibitors of both VEGFR and EGFR, and Glivec. The further drug may alternatively be paclitaxel or an analogue, or other microtubule inhibitor, a topoisomerase inhibitor.

Combinations of rapamycin with other anticancer agents is also known, and the known combination disclosed below may be used in this aspect of the invention. Cisplatin, gemcitabine, tamoxifen, interferon alpha, EGFR inhibitor, VEGFR/EGFR inhibitor and Gleevec have all been combined with rapamycin analogues in preclinical or clinical studies, and these combinations may be used in the further aspect of the invention.

Interactions between rapamycin and chemotherapeutic agents used as first- and second-line agents against breast cancer have been investigated. In vitro, synergistic interactions were observed in combinations with paclitaxel, carboplatin, and vinorelbine. Additive effects were observed in combinations with doxorubicin and gemcitabine. Rapamycin dramatically enhanced paclitaxel- and carboplatin-induced apoptosis. This effect was sequence dependent and mediated at least partly through caspase activation. Furthermore, rapamycin enhanced chemosensitivity to paclitaxel and carboplatin in HER2/neu-overexpressing cells, suggesting a potential approach to these poorly behaving tumours. In vivo, rapamycin combined with paclitaxel resulted in a significant reduction in tumour volume compared with either agent alone in rapamycin-sensitive tumours. (Mondesire W H, et al., 2004, Clinical Cancer Research, 10, 7031-7042). The combination of a rapamycin or analogue with a taxane may be used in the further aspect of the invention.

RAD001 dramatically enhances cisplatin-induced apoptosis in wt p53, but not mutant p53 tumour cells. RAD001 sensitises cells to cisplatin by inhibiting p53-induced p21 expression. These findings support the molecular rationale for combining DNA damaging agents such as the platinum cytotoxic with RAD001, showing that a general major anabolic process may enhance the efficacy of an established drug protocol in the treatment of patients with solid tumours (Beuvink I et al 2005, Cell, 120:747-759). The combination of rapamycin or analogues with a platinum anti-neoplastic compound may be used in the further aspects of the invention.

CCI-779 and tamoxifen or another anti-estrogen may be useful if administered in combination. The PI3K/AKY/mTOR pathway may play an important role in the development of tamoxifen resistance in breast cancer. Activation of the estrogen receptor can drive the PI3K pathway. Cross talk between erb-1 and ER has been shown to activate the pathway, which has been associated with estrogen-independent transcriptional activity, and breast cancer cell lines with activated AKT are resistant to the growth inhibitory effects of tamoxifen. The combination of rapamycin or an analogue with an anti-estrogen may be used in this further aspect of the invention.

There is an ongoing phase III trial comparing orally administered temsirolimus (CCI-779) plus letrozole (an aromatase inhibitor) versus letrozole alone as first-line treatment among approximately 1200 postmenopausal women with advanced breast cancer. The combination of rapamycin or an analogue with an aromatase inhibitor may be used in this further aspect of the invention.

CCI-779 and interferon alpha have been used in conjunction in treatment of renal cell carcinoma (RCC). A large phase 3 trial is ongoing. The combination of rapamycin or an analogue and alpha interferon may be used in this further aspect of the invention.

Rapamycin with epidermal growth factor receptor (EGFR) inhibitor EKI-785 resulted in synergistic growth inhibition. Similar results were achieved with RAD-001 and VEGFR/EGFR inhibitor AEE788. Rapamycin with Glivec resulted in synergistic growth inhibition of CML cell lines. The combination of rapamycin or an analogue with a growth factor receptor or with Glivec may be used in this further aspect of the invention.

Other combinations used in the further aspect of the invention include cytotoxic agents with P-glycoprotein inhibitors. Approximately 50% of cancer patients receive chemotherapy, and as many as 75% of these patients experience intrinsic or acquired resistance to a broad spectrum of chemotherapeutic agents. This phenomenon, termed multidrug resistance (MDR), is the most common cause of chemotherapy failure. It is well established that the majority of tumours develop MDR through over-expression of the drug efflux pump P-glycoprotein (P-gp). Inhibition of P-gp is acknowledged as a viable means of reversing MDR; however, existing P-gp inhibitors so far have demonstrated limited clinical success due to limitations in potency and specificity. Verapamil, a calcium channel blocker, is commonly used for the treatment of supraventricular arrhythmias, coronary heart disease, and arterial hypertension. Furthermore, verapamil is a potent inhibitor of P-glycoprotein-mediated transport, and has been proven to modify MDR with cancer chemotherapy in in vitro experiments (Toffoli G, et al. 1995, Biochem Pharmacol, 50, 1245-55; Pauli-Magnus C, et al. 2000, J Pharmacol Exp Ther, 293, 376-82). Local delivery of doxorubicin and verapamil from jointly loaded drug eluting beads may therefore offer a synergistic benefits, allowing high local doses of cytotoxic agent and P-gp inhibition to overcome any MDR effects. Thus the combination of a cytotoxic antibiotic and a P-gp inhibitor may be used in the further aspect of this invention.

Other combinations include topoisomerase inhibitors, such as tecans with other actives having low Combination Index.

Other combinations as described by Celator in any of WO2003028696, WO2004087115, WO2004087105, WO2004093795, WO2006055903, and in Tardi, P. G. et al., (2007) Biochim. Biophys. Acta 1768, 678-587, the contents of which are incorporated herein by reference, may be used in the further aspect of the invention.

Taxanes and anti-metabolites may be used in combination, such as paclitaxel and 5-FU. 5FU and leucovorin may be used.

Where the actives include a camptothecin derivative, it is particularly effective to use a derivative having cationic substituents, such as compounds of the general formula I

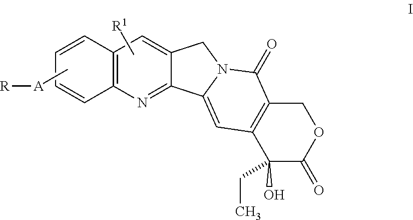

in which R¹ is selected from H, halogen, hydroxyl and lower (C₁₋₆) alkyl, optionally substituted by a hydroxyl, amine, alkoxy, halogen, acyl and acyloxy groups;

### A is C(O)O or CH₂; and

R is NR²R³ where R² and R³ are the same or different and each represents a hydrogen atom, a substituted or unsubstituted C₁₋₄ alkyl group or a substituted or unsubstituted carbocyclic or heterocyclic group, or R² and R³ together with the nitrogen atom to which they are attached from a optionally substituted heterocyclic ring which may be interrupted by —O—, —S— or >NR⁴ in which R⁴ is a hydrogen atom, a substituted or unsubstituted C₁₋₄ alkyl group or a substituted or unsubstituted phenyl group; and wherein the grouping -A-R is bonded to a carbon atom located in any of the 9, 10 or 11 positions in the A ring of the camptothecin compound and R¹ is substituted in the A or B ring, including salts thereof.

In one embodiment the camptothecin derivatives have the formula IA

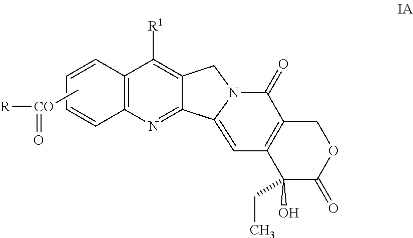

in which R¹ is H, lower (C₁₋₆) alkyl, optionally substituted by a hydroxyl, amine, alkoxy, halogen, acyl or acyloxy group or halogen; and

R is NR²R³ where R² and R³ are the same or different and each represents a hydrogen atom, a substituted or unsubstituted C₁₄ alkyl group or a substituted or unsubstituted carbocyclic or heterocyclic group, or R² and R³ together with the nitrogen atom to which they are attached from a optionally substituted heterocyclic ring which may be interrupted by —O—, —S— or >NR⁴ in which R⁴ is a hydrogen atom, a substituted or unsubstituted C₁₋₄ alkyl group or a substituted or unsubstituted phenyl group;

and wherein the grouping —O—CO—R is bonded to a carbon atom located in any of the 9, 10 or 11 positions in the A ring of the camptothecin compound, including salts thereof.

It is preferred for the grouping —O—CO—R to be joined at the 10 position.

R¹ is preferably C₁₋₄ alkyl, most preferably ethyl, and m is preferably 1.

A halogen atom R is, for instance, F, CI, Br or I, preferably F or Cl. R¹ to R⁴ may be methyl, ethyl, propyl, isopropyl, in-butyl, isobutyl and t-butyl, preferably methyl.

Substituents in R and R¹ are preferably selected from halogen atoms, hydroxy, C₁₋₄ alkoxy, phenoxy, COOR⁶, SO₃R⁶ and PO₃(R⁶)₂, aryl,

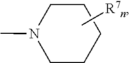

NR⁸R⁹ and CONR⁸R⁹, QAOR⁵, QANR⁸R⁹ and QAQR⁵ in which R⁵ is C₁₋₄ alkyl or aryl; R⁶ is hydrogen, halogen C₁₋₄ alkyl or C₁₋₄ alkoxy; R⁷ is hydrogen, halogen or C₁₋₄ alkyl; R⁸ and R⁹ are the same or different and each is H, or C₁₋₄ alkyl or R⁸ and R⁹ together represent C₃₋₆ alkanediyl;

Q is OCO, or —COO— and A is C₂₋₄ alkanediyl.

Preferably R is NR³, R³ where R² and R³ together with the nitrogen atom form a 5 or 6 membered ring, preferably a saturated ring, with optional substituents. A substituent is preferably —NR⁸R⁹. In such a substituent R⁸ and R⁹ preferably together are C₄₋₅ alkanediyl. Such groups are basic and tend to be cationically charged at pH7. Most preferably R is

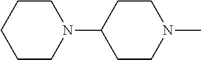

Another family of suitable compounds has the general formula II

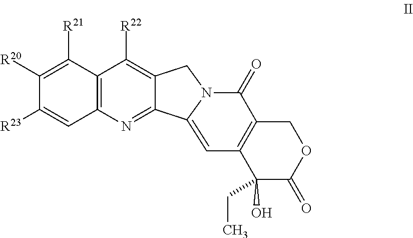

in which R²⁰ and R²³ are each hydroxy or hydrogen or together are CH₂OCH₂;

one of R²¹ and R²² is H and the other is CH₂NR²⁴R²⁵ where R²³ and R²⁴ are the same or different and each represents a hydrogen atom, a substituted or unsubstituted C₁₋₄ alkyl group or a substituted or unsubstituted carbocyclic or heterocyclic group, or R²³ and R²⁴ together with the nitrogen atom to which they are attached from a optionally substituted heterocyclic ring which may be interrupted by —O—, —S— or >NR⁴ in which R⁴ is a hydrogen atom, a substituted or unsubstituted C₁₋₄ alkyl group or a substituted or unsubstituted phenyl group; including salts and quaternary derivatives thereof. One example of a suitable compound of this claim is topotecan, in which R²⁰ is hydroxyl, R²² and R²³ are hydrogen, R²¹ is CH₂NR²⁴R²⁵ and R²⁴ and R²⁵ are both methyl.

The ratio of the first active to the second active in both the above aspects of the invention is preferably in the range 100:1 to 1:100, preferably in the range 5:1 to 1:5. Suitable ratios may be determined by a person skilled in the art, for instance by using information available from combination index determinations. Generally the ratios should be selected so as to optimise the synergistic effect.

Preferably the ratio of equivalents of anion in the polymer to cation in the first and, where it is cationically charged, second active, should be in the range 2:1 to 1:1, preferably around 1:1. By selecting these ratios optimum loading may be achieved, while minimising the first effect upon initial administration, and providing optimised extended release.

The second active used in the first and second aspects of the invention may alternatively be a COX-inhibitor. COX-inhibitors, for instance NSAIDs, could act as anti-inflammatory and analgesic agents, targeting inflammation and pain associated with the chemoembolisation procedure. Furthermore, up to 50% of the cells in a tumour can be inflammatory cells. Also there have been numerous studies linking inflammation with angiogenesis and tumour progression. Accordingly a combination of a cytotoxic agent such as doxorubicin, with a COX-inhibitor may have highly desirable effects. As indicated above, ibuprofen has even been proposed as having useful effects on reducing cancer cell invasion and liver metastases.

The polymer is a water-insoluble material. Although it may be biodegradable, so that drug may be released substantially by erosion of polymer matrix to release drug from the surface, preferably the polymer is substantially biostable (i.e. non-biodegradable).

The polymer is water-swellable. Water-swellable polymer useful in the invention preferably has an equilibrium water content, when swollen in water at 37° C., measured by gravimetric analysis, in the range of 40 to 99 wt %, preferably 75 to 95%.

Generally the polymer is covalently crosslinked, although it may be appropriate for the polymer to be ionically crosslinked, at least in part, or hydrophobically crosslinked. In some embodiments it may be suitable to use polymers which are derived from natural sources, such as albumin, alginate, gelatin, starch, chitosan or collagen, all of which have been used as embolic agents. In other embodiments the polymer is substantially free of naturally occurring polymer or derivatives. It is preferably formed by polymerising ethylenically unsaturated monomers in the presence of di- or higher-functional crosslinking monomers. The ethylenically unsaturated monomers may include an ionic (including zwitterionic) monomer.

Copolymers of hydroxyethyl methacrylate, acrylic acid and cross-linking monomer, such as ethylene glycol dimethacrylate or methylene bisacrylamide, as used for etafilcon A based contact lenses may be used. Copolymers of N-acryloyl-2-amino-2-hydroxymethyl-propane-1,3-diol and N,N-bisacrylamide may also be used.

Other suitable synthetic polymers are cross-linked styrenic polymers e.g. with ionic substituents, of the type used as separation media or as ion-exchange media.

Another type of synthetic polymer which may be used to form the water-swellable water-insoluble matrix is polyvinyl alcohol crosslinked using aldehyde-type crosslinking agents such as glutaraldehyde. For such products, the polyvinyl alcohol (PVA) may be rendered ionic by providing pendant ionic groups by reacting a functional ionic group containing compound with the hydroxyl groups. Examples of suitable functional groups for reaction with the hydroxyl groups are acylating agents, such as carboxylic acids or derivatives thereof, or other acidic groups which may form esters.

The invention is of particular value where the polymer matrix is formed from a polyvinyl alcohol macromer, having more than one ethylenically unsaturated pendant group per molecule, by radical polymerisation of the ethylenic groups. Preferably the PVA macromer is copolymerised with ethylenically unsaturated monomers for instance including a nonionic and/or ionic monomer including anionic monomer.

The PVA macromer may be formed, for instance, by providing PVA polymer of a suitable molecular weight, such as in the range 1000 to 500,000 D, preferably 10,000 to 100,000 D, with pendant vinylic or acrylic groups. Pendant acrylic groups may be provided, for instance, by reacting acrylic or methacrylic acid with PVA to form ester linkages through some of the hydroxyl groups. Other methods for attaching vinylic groups capable of polymerisation onto polyvinyl alcohol are described in, for instance, U.S. Pat. No. 4,978,713 and, preferably, U.S. Pat. Nos. 5,508,317 and 5,583,163. Thus the preferred macromer comprises a backbone of polyvinyl alcohol to which is linked, via a cyclic acetal linkage, an (alk)acrylaminoalkyl moiety. Example 1 describes the synthesis of an example of such a macromer known by the approved name nelfilcon B. Preferably the PVA macromers have about 2 to 20 pendant ethylenic groups per molecule, for instance 5 to 10.

Where PVA macromers are copolymerised with ethylenically unsaturated monomers including an ionic monomer, the ionic monomer preferably has the general formula II

Y¹BQ¹ II

in which Y¹ is selected from

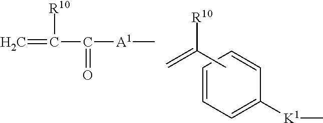

CH₂═C(R¹⁰)—CH₂—O—, CH₂═C(R¹⁰)—CH₂OC(O)—, CH₂═C(R¹⁰)OC(O)—, CH₂═C(R¹⁰)—O—, CH₂═C(R¹⁰)CH₂OC(O)N(R¹¹)—, R¹²OOCCR¹⁰═CR¹⁰C(O)—O—, R¹⁰CH═CHC(O)O—, R¹⁰CH═C(COOR¹²)CH₂—C(O)—O—,

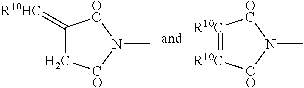

wherein:

R¹⁰ is hydrogen or a C₁-C₄ alkyl group;

R¹¹ is hydrogen or a C₁-C₄ alkyl group;

R¹² is hydrogen or a C₁₋₄ alkyl group or BQ¹ where B and Q¹ are as defined below;

A¹ is —O— or —NR¹¹—;

K¹ is a group —(CH₂)ᵣOC(O)—, —(CH₂)ᵣC(O)O—, —(CH₂)ᵣOC(O)O—, —(CH₂)ᵣNR¹³—, —(CH₂)ᵣNR¹³C(O)—, —(CH₂)ᵣC(O)NR¹³—, —(CH₂)ᵣNR¹³C(O)O—, —(CH₂)ᵣOC(O)NR¹³—, —(CH₂)ᵣNR¹³C(O)NR¹³— (in which the groups R¹³ are the same or different), —(CH₂)ᵣO—, —(CH₂)ᵣSO₃—, or, optionally in combination with B, a valence bond and r is from 1 to 12 and R¹³ is hydrogen or a C₁-C₄ alkyl group;

B is a straight or branched alkanediyl, oxaalkylene, alkanediyloxaalkanediyl, or alkanediyloligo(oxaalkanediyl) chain optionally containing one or more fluorine atoms up to and including perfluorinated chains or, if Q¹ or Y¹ contains a terminal carbon atom bonded to B a valence bond; and

Q¹ is an anionic group.

An anionic group Q¹ may be, for instance, a carboxylate, carbonate, sulphonate, sulphate, nitrate, phosphonate or phosphate group. The monomer may be polymerised as the free acid or in salt form. Preferably the pKₐ of the conjugate acid is less than 5. In the further aspect of the invention Q¹ may be a cationic group, a zwitterionic group, or a nonionic group.

In the further aspect of the invention Q¹ may be a cationic group, a zwitterionic group, or a non-ionic group.

A suitable cationic group Q¹ is preferably a group N⁺R¹⁴₃, P⁺R¹⁵₃ or S⁺R¹⁵₂ in which the groups R¹⁴ are the same or different and are each hydrogen, C₁₋₄-alkyl or aryl (preferably phenyl) or two of the groups R¹⁴ together with the heteroatom to which they are attached from a saturated or unsaturated heterocyclic ring containing from 5 to 7 atoms the groups R¹⁵ are each OR¹⁴ or R¹⁴. Preferably the cationic group is permanently cationic, that is each R¹⁴ is other than hydrogen. Preferably a cationic group Q is N⁺R¹⁴₃ in which each R¹⁴ is C₁₋₄-alkyl, preferably methyl.

A zwitterionic group Q¹ may have an overall charge, for instance by having a divalent centre of anionic charge and monovalent centre of cationic charge or vice-versa or by having two centres of cationic charge and one centre of anionic charge or vice-versa. Preferably, however, the zwitterion has no overall charge and most preferably has a centre of monovalent cationic charge and a centre of monovalent anionic charge.

Examples of zwitterionic groups which may be used as Q¹ in the present invention are disclosed in WO-A-0029481.

Where the ethylenically unsaturated monomer includes zwitterionic monomer, for instance, this may increase the hydrophilicity, lubricity, biocompatibility and/or haemocompatibility of the particles. Suitable zwitterionic monomers are described in our earlier publications WO-A-9207885, WO-A-9416748, WO-A-9416749 and WO-A-9520407. Preferably a zwitterionic monomer is 2-methacryloyloxy-2′-trimethylammonium ethyl phosphate inner salt (MPC).

In the monomer of general formula I preferably Y¹ is a group CH²═CR¹⁰COA— in which R¹⁰ is H or methyl, preferably methyl, and in which A¹ is preferably NH. B is preferably an alkanediyl group of 1 to 12, preferably 2 to 6 carbon atoms. Such monomers are acrylic monomers.

There may be included in the ethylenically unsaturated monomer diluent monomer, for instance non-ionic monomer. Such a monomer may be useful to control the pKₐ of the acid groups, to control the hydrophilicity or hydrophobicity of the product, to provide hydrophobic regions in the polymer, or merely to act as inert diluent. Examples of non-ionic diluent monomer are, for instance, alkyl (alk) acrylates and (alk) acrylamides, especially such compounds having alkyl groups with 1 to 12 carbon atoms, hydroxy, and di-hydroxy-substituted alkyl(alk) acrylates and -(alk) acrylamides, vinyl lactams, styrene and other aromatic monomers.

In the polymer matrix in the first aspect of the invention and in a preferred embodiment of the further aspect of the invention, the level of anion is preferably in the range 0.1 to 10 meq g⁻¹, preferably at least 1.0 meq g⁻¹. Preferred anions are derived from strong acids, such as sulphates, sulphonates, phosphates and phosphonates.

Where PVA macromer is copolymerised with other ethylenically unsaturated monomers, the weight ratio of PVA macromer to other monomer is preferably in the range of 50:1 to 1:5, more preferably in the range 20:1 to 1:2. In the ethylenically unsaturated monomer the anionic monomer is preferably present in an amount in the range 10 to 100 mole %, preferably at least 25 mole %.

The crosslinked polymer is formed as such in microsphere form, for instance by polymerising in droplets of monomer in a dispersed phase in a continuous immiscible carrier. Examples of suitable water-in-oil polymerisations to produce particles having the desired size, when swollen, are known. For instance U.S. Pat. No. 4,224,427 describes processes for forming uniform spherical beads (microspheres) of up to 5 mm in diameter, by dispersing water-soluble monomers into a continuous solvent phase, in a presence of suspending agents. Stabilisers and surfactants may be present to provide control over the size of the dispersed phase particles. After polymerisation, the crosslinked microspheres are recovered by known means, and washed and optionally sterilised. Preferably the particles e.g. microspheres, are swollen in an aqueous liquid, and classified according to their size.

Methods for loading the two actives into a microsphere are selected according to the nature of the actives and the loading levels required. Where both the actives are cationically charged and the matrix is anionic, the two actives may be loaded sequentially or simultaneously. Where they are loaded simultaneously, this is suitable from a solution containing both the actives in combination with one another. Sequential loading methods may involve contact of one of the actives in an aqueous solution with polymer at a level or for a time such that ion-exchange within the polymer matrix takes place to an extent such that a proportion of the anionic groups of the polymer remain unassociated with cationic active. In a second step, optionally after removal of non-absorbed first active, a solution of the second active, usually in water, is contacted with the partially loaded microspheres for a period of time allowing ion-exchange to take place and the second drug to become loaded into the polymer.

Where one of the actives is a compound having low water-solubility, a different technique is used. For instance it may be possible to load the actives sequentially, for instance by contacting the microspheres with solutions of each drug in suitable solvents, enabling the microspheres to swell and absorb the active into the polymer matrix. In one embodiment, a low water-solubility active is loaded into the polymer first. In another embodiment, the cationically charged active is loaded first.

It may be possible to select a solvent or solvent system in which both a cationically charged active and a neutral active, which has low water-solubility, are soluble, the solvent being one which acts to swell the polymer. For instance, where the actives are both soluble in selected alcohol, the alcohol may be used to dissolve the actives together, and to swell the polymer matrix which is swellable in the selected alcohol.

Preferably the loading process involves the steps:

contacting microspheres which comprise a matrix of a water-insoluble water-swellable anionically charged crosslinked polymer in substantially dry form with a solution of a drug having a water-solubility less than 10 g/l at room temperature in a first organic solvent, whereby a solution of drug in solvent becomes impregnated into the microspheres;

separating drug solution which has not impregnated the microspheres;

contacting the impregnated microspheres with aqueous liquid whereby drug is precipitated in the core of the microspheres;

contacting the microspheres containing precipitated drug with an aqueous solution of an ionic drug having a charge opposite to that of the polymer to allow loading of ion-exchange; and

recovering the dual loaded microspheres, wherein the drugs are selected as in the first or further aspects of the invention.

In this method, we have found that we can achieve high levels of loading for both the water-insoluble drug and the cationic drug. The level of loading of cationic drug is as high as for the neat microspheres. Where the loading method is carried out with the ionic drug being loaded first and the neutral water-insoluble drug being loaded second, the possible loading levels achieved for the second, neutral drug are lower than for neat microspheres. This effect is further shown in Example 4 below.

We believe it is the first time that a water-swellable ionically charged drug delivery polymer has been loaded with a combination of a water-insoluble drug and a drug having an opposite charge to the polymer. According to a yet further aspect (referred to hereinafter as the third aspect) of the invention a drug delivery matrix which comprises a water-insoluble, water-swellable ionically charged polymer which is swellable in water at room temperature to an equilibrium water content in the range 40 to 99% by weight based on polymer plus water is contacted with a solution of drug having a water-solubility less than 10 g/l at room temperature in an organic solvent which is capable of swelling the polymer, whereby the solution becomes impregnated into the matrix;

any drug solution which has not impregnated the polymer matrix is separated;

the impregnated matrix is contacted with water whereby drug is precipitated in the matrix;

the matrix having precipitated water-insoluble drug is contacted with an aqueous solution of the ionically charged drug whereby the matrix swells and ion-exchange with counterions associated with the polymer takes place and drug is electrostatically bound in the polymer matrix.

Optionally the matrix loaded with the low solubility drug is subsequently rinsed and/or dried. The subsequent steps up to contact with the aqueous solution of the ionically charged drug may be as described in WO2007090897 the content of which is incorporated herein by reference and the preferred components and conditions described in that specification may be used for the first steps in the process of this embodiment, including the drugs. The matrix may be in the form of a microsphere according to the first aspect of the invention. Alternatively it may be a drug delivery device article such as an implant in the form of a needle, slab, disc, thread or film of polymer. Alternatively it is a coating on a device which is implanted such as a stent, catheter, needle, tubing, graft, valve, wire, etc.

The polymer may be an anionic polymer as used in the first aspect of the invention described above. Alternatively it may be a cationic polymer, for instance also having zwitterionic pendant groups, as described in WO0152915, the content of which is incorporated herein by reference. Alternatively it may be another ionic hydrogel polymer used in drug delivery device such as a dextran sulphate-based material as described in Liu et al., (2003) op. cit.

Preferably it is synthetic, although it may alternatively be a natural-based polymer, based on a protein or a polysaccharide, such as collagen, hyaluronic acid, dextran sulphate, alginate, a cellulosic polymer or albumin.

In the method of the third aspect of the invention the matrix loaded with the first drug may be dried before contact with the aqueous solution of the second drug. After ion-exchange with the second drug excess drug solution may be removed before rinsing. This aspect of the invention is of particular value where the water-insoluble drug is rapamycin or an analogue as described above, or a taxane such as paclitaxel, or dexamethasone.

In an alternative embodiment of loading method to produce the product of the first or further (second) aspect of the invention, the active which is not charged may be a material which is crystalline at room temperature, but for which it would be desirable to avoid being in crystalline form. Such drugs include, for instance, ibuprofen. For these drugs, crystallisation of the drug may be inhibited by incorporating a crystallisation inhibitor, such as an oil, for instance a mineral oil or a vegetable oil, optionally an oil which acts as an imaging agent, for instance lipiodol or another iodinated oil. Suitably the process involves contacting microspheres comprising a matrix of a water-insoluble water-swellable ionically charged crosslinked polymer with a solution of the crystallisable drug in an organic solvent capable of penetrating the microspheres, the solution further containing a crystal modifier which inhibits crystallisation of the drug, and, in a subsequent step, contacting the microspheres loaded with crystallisable drug with an aqueous solution of an ionic drug with a charge opposite that of the polymer matrix in an amount and for a time suitable for loading the drug by ion-exchange. Preferably in the method, following loading of the crystallisable drug, the beads are washed with water or an aqueous solution to remove non-absorbed crystallisable drug.

In the invention of the first or further (second or third) aspects of the invention, the microspheres may be suitable for administration in an embolisation method. The microspheres are therefore of a size which renders them suitable for use in embolisation, depending on the animal being treated, and the location of the tumour, and thus the size of the feeding blood vessels. For instance embolic microspheres preferably have a size when swollen in deionised water at room temperature (neat) of 40 μm up to 1500 μm, preferably in the range 100 μm up to 1200 μm. Alternatively the microspheres may be suitable for administration as a drug depot, for instance which may release drug over a long period into the surrounding tissue, and optionally into the circulation. In another embodiment, the microspheres are suitable for intratumoural injection. Microspheres having sizes in the range mentioned above for embolisation may also be suitable for intratumoural administration or for administration in the resection margins following tumour resection.

Methods for loading cationically charged and water-insoluble actives into anionic matrix polymers as described above for use with microspheres, may also be applicable for loading such drug types into other hydrogel based drug delivery articles. There is therefore provided in a fourth aspect of the invention a new drug delivery article comprising a water-swellable matrix formed of water-insoluble, water-swellable polymer which is ionically charged, and, incorporated into the matrix, a first active which is oppositely charged, and a second active which is a crystallisable compound, in which the matrix is contacted with a solution of the crystallisable drug in a volatile organic solvent capable of swelling the matrix further containing a crystal modifier capable of inhibiting crystallisation of the drug, drying the matrix to remove the volatile solvent, contacting the matrix loaded with crystallisable drug and crystal modifier with an aqueous solution of the ionic drug whereby the matrix swells and ion-exchange with counterions associated with the polymer takes place and drug is electrostatically bound in the polymer matrix.

Steps subsequent to the drying to remove solvent may be described above for the third method aspect of the invention. The polymers that are suitable are also as described above in connection with the third method aspect as well as the drug delivery articles for which this aspect may be used. The first steps in the process and the solvents and conditions may be as further described in WO2007073688, the content of which are incorporated herein by reference. That specification also describes drugs for which this aspect of the invention is suited as the crystallisable drug.

The drug delivery article in this (fourth) aspect of the invention may be a microsphere, for instance for use in tumour treatment, especially for use in a method of chemoembolisation. Alternatively the article may be a microimplant, for instance in the form of a needle, disc, slab, or other shape, preferably which may be administered by injection, for instance through a needle, preferably through a catheter. The article may be provided with the polymer as a coating, and may comprise a substrate formed of a glass, plastics or metal material. For instance the article may be a catheter or an implant for instance a prosthetic device, a cardiovascular stent or any of the devices described above in connection with the second aspect of the invention.

In the third and fourth aspects of the invention the two drugs are preferably selected so as to be therapeutically effective in tumour treatment, and preferably have a combination index as hereinbefore defined of less than 0.9, preferably less than 0.7, for instance less than 0.5, preferably less than 0.3, and most preferably less than 0.1.

The ionic drug in the third and fourth aspect of the invention may be a cationic anthracycline as described in WO04071495, the contents of which are incorporated herein by reference, or a camptothecin derivative such as described in WO2006027567. Alternatively the drug may be anionic such as described in WO0152915 to contents of which are incorporated herein by reference.

The present invention is illustrated in the accompanying examples. The examples refer to various figures which relate briefly to the following results:

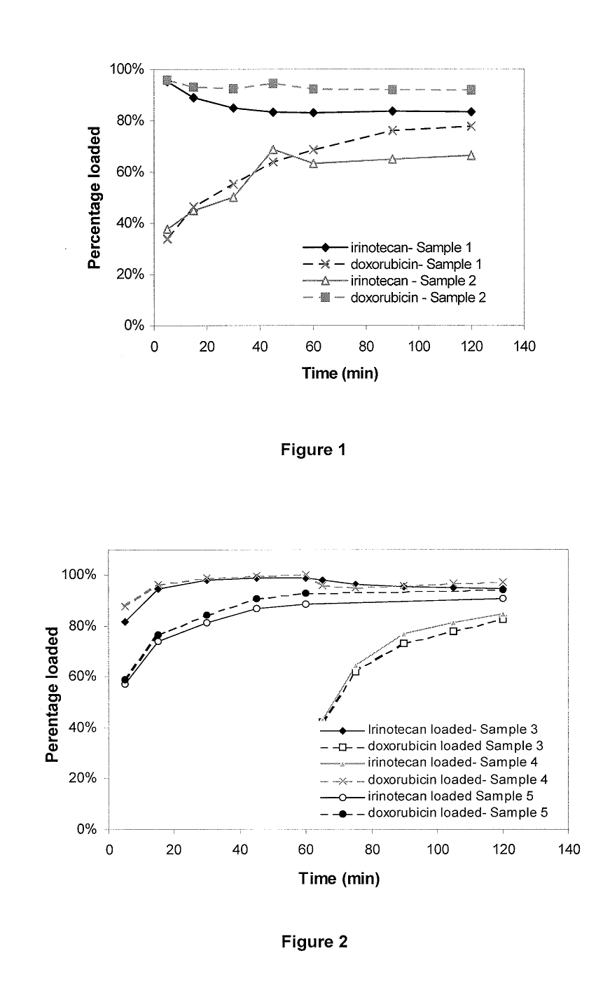

FIG. 1 shows the loading profile of beads loaded with doxorubicin (Sample 1) or irinotecan (Sample 2) at a level of 25 mg.ml⁻¹ of beads after loading with the second drug at a saturation level, n=1, as described in Example 2;

FIG. 2 shows the loading profile of irinotecan and doxorubicin combined into beads. Loading profile of beads loaded with 25 mg of irinotecan for 1 h and then with 25 mg of doxorubicin (Sample 3), or with 25 mg of doxorubicin for 1 h and then with 25 mg of irinotecan (Sample 4) and loading profile of beads loaded with a mixture of 25 mg of doxorubicin and 25 mg of irinotecan (Sample 5), n=1, as described in Example 2;

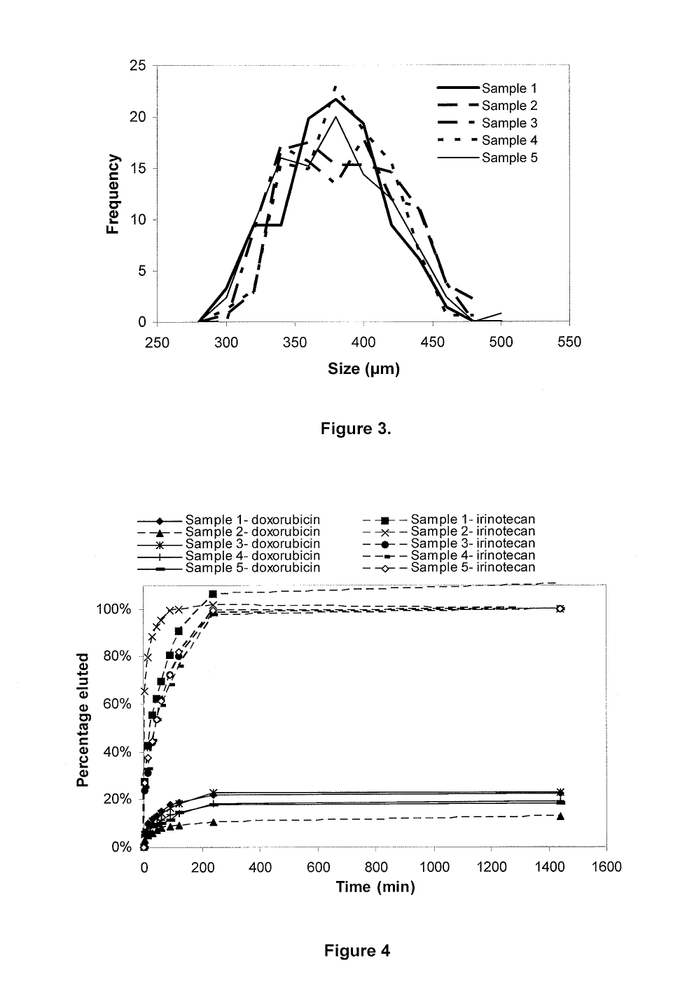

FIG. 3 shows the size of samples loaded with a combination of doxorubicin and irinotecan by different methods, n=1, as described in Example 2;

FIG. 4 shows the elution profile of doxorubicin and irinotecan eluted into PBS at RT from the same beads. Sample 1 is beads loaded with doxorubicin at a level of 25 mg.ml⁻¹ of beads after loading with irinotecan at a saturation level, Sample 2 is beads loaded with irinotecan at a level of 25 mg ml⁻¹ of beads after loading with doxorubicin at a saturation level. Sample 3 is beads loaded with 25 mg of irinotecan for 1 h and then with 25 mg of doxorubicin, Sample 4 is beads loaded with 25 mg of doxorubicin for 1 h and then with 25 mg of irinotecan and Sample 5 is beads loaded with a mixture of 25 mg of doxorubicin and 25 mg of irinotecan, n=1, as described in Example 2;

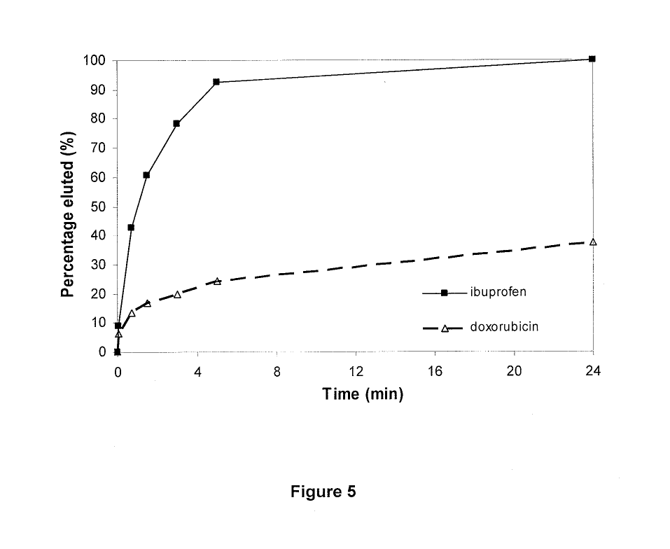

FIG. 5 shows the release profile of microspheres loaded with doxorubicin and ibuprofen into 200 ml of PBS at RT onto a roller-mixer for 24 h, n=1, as described in Example 3;

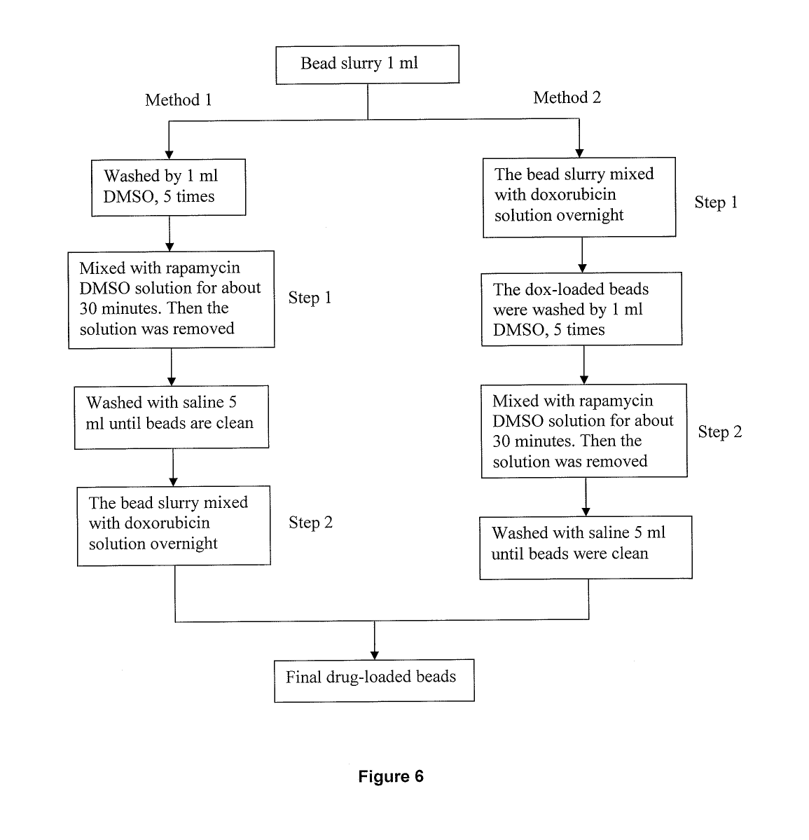

FIG. 6 is a flow diagram showing two methods of rapamycin and doxorubicin loading into beads, as described in Example 4;

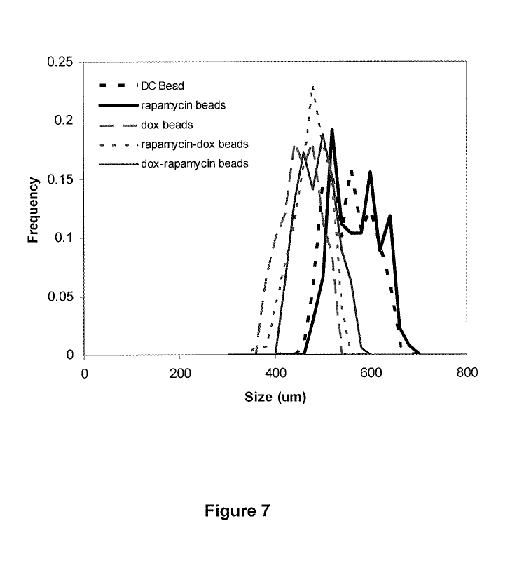

FIG. 7 shows the size distribution of Unloaded and drug-loaded Beads, as described in Example 4;

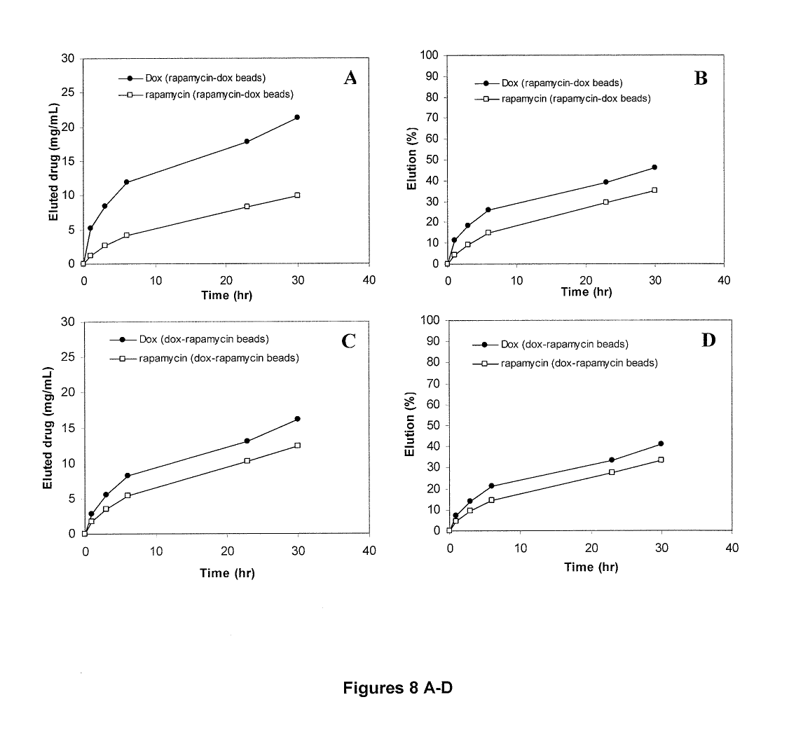

FIGS. 8 A-D show doxorubicin and rapamycin elution from loaded beads to 200 mL PBS, 25° C. (A) Drug elution from the beads made by Method 1. (B) Elution data of (A) presented as percentage. (C) Drug elution from the beads made by Method 2. (D) Elution data of (C) presented as percentage. In (A) and (C), the amount of drug eluted were corrected to per volume of beads without drug, as described in Example 4;

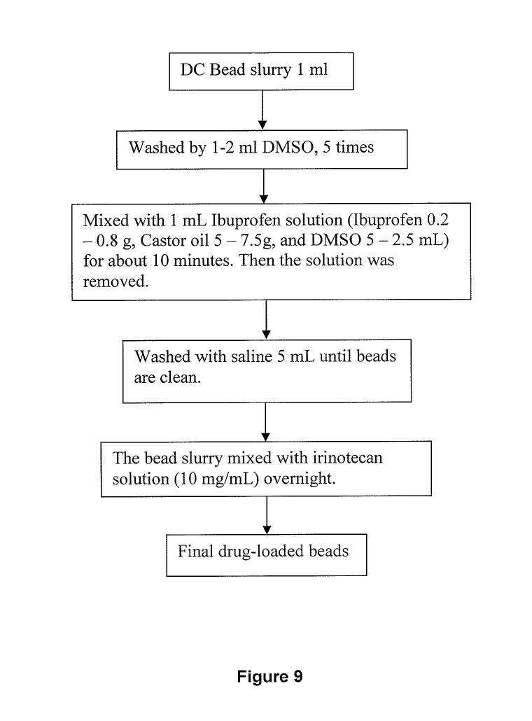

FIG. 9 shows the procedure of ibuprofen-irinotecan bead preparation, as described in Example 5;

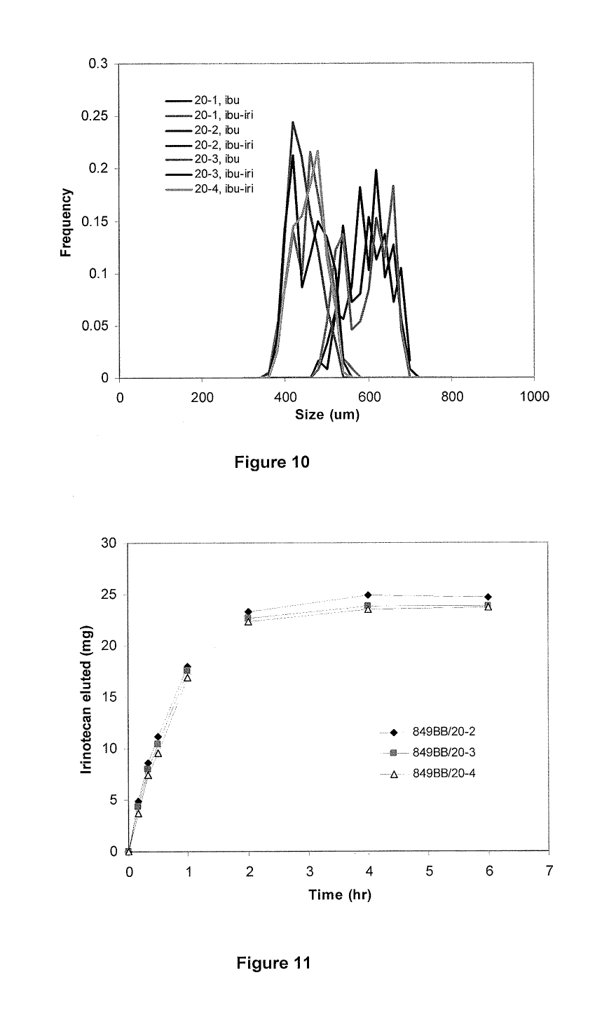

FIG. 10 shows the size distribution of ibuprofen- and ibuprofen-irinotecan-loaded beads, as described in Example 5;

FIG. 11 shows the elution of irinotecan from beads with the combination of ibuprofen and irinotecan. Elution condition: 0.24 ml beads (loaded), 200 mL PBS, 25° C., as described in Example 5;

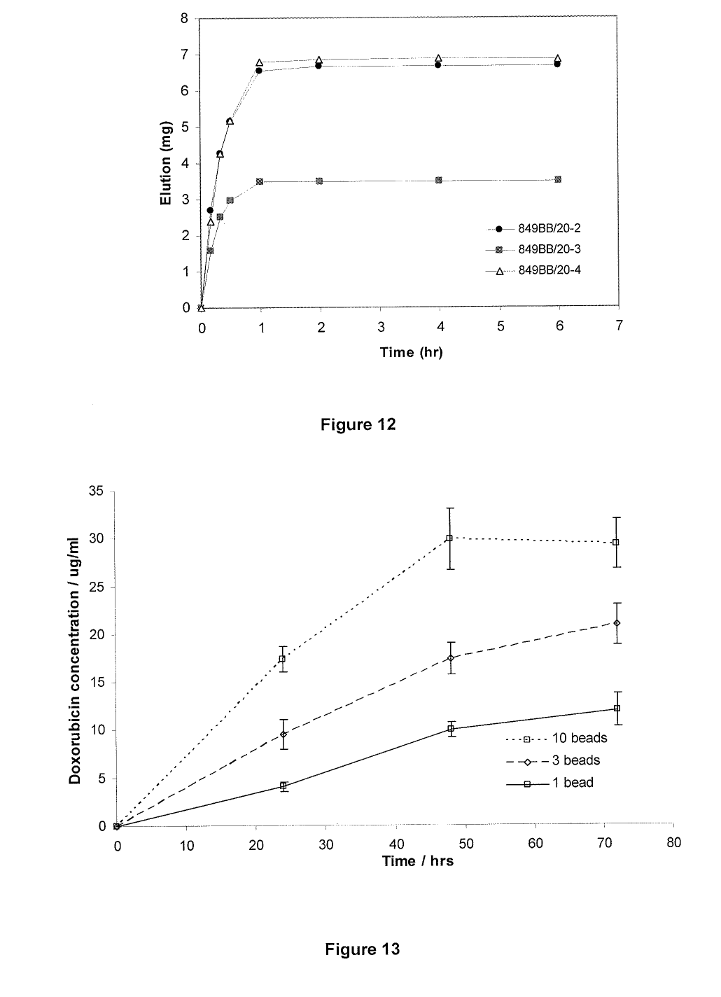

FIG. 12 shows ibuprofen elution from beads with the combination of ibuprofen and irinotecan. Elution condition: 0.24 mL beads (loaded), 200 ml PBS, 25° C., as described in Example 5;

FIG. 13 shows doxorubicin elution from Beads vs. elution time. (Error bars: 1SD, n=6), as described in Example 6;

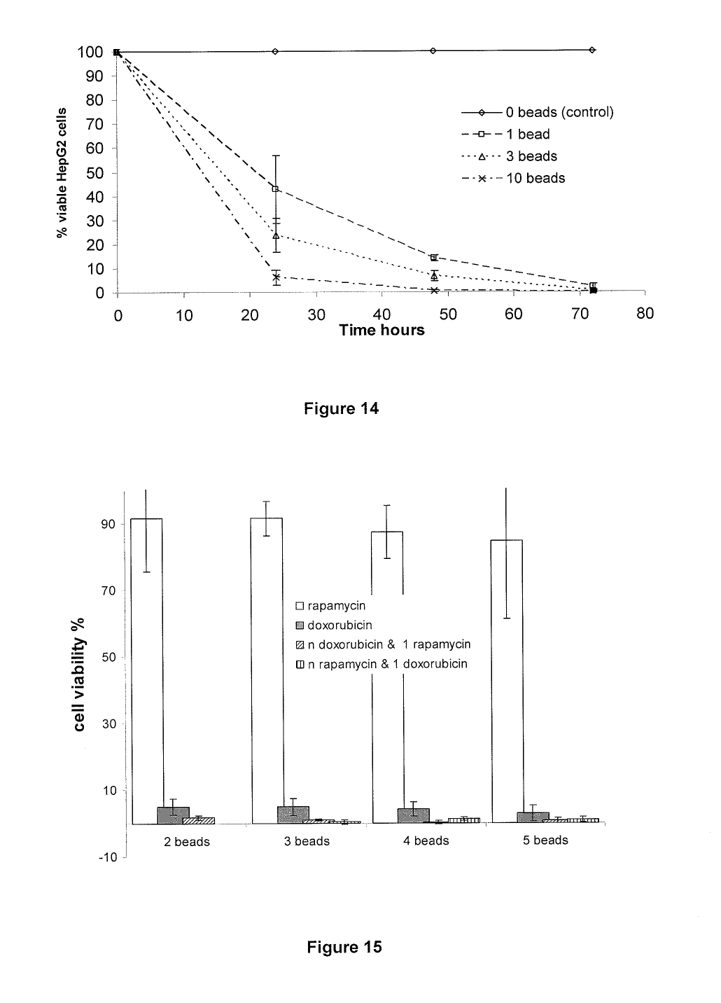

FIG. 14 shows the viable HepG2 cells percentage as compared to untreated cells with respect to exposure time and bead number from LDH assay. (Error bars: 1SD, n=6), as described in Example 6;

FIG. 15 shows the percentage of viable HepG2 cells for rapamycin, doxorubicin and combination (Error bars: 1SD, n=5), results from LDH assay, as described in Example 7;

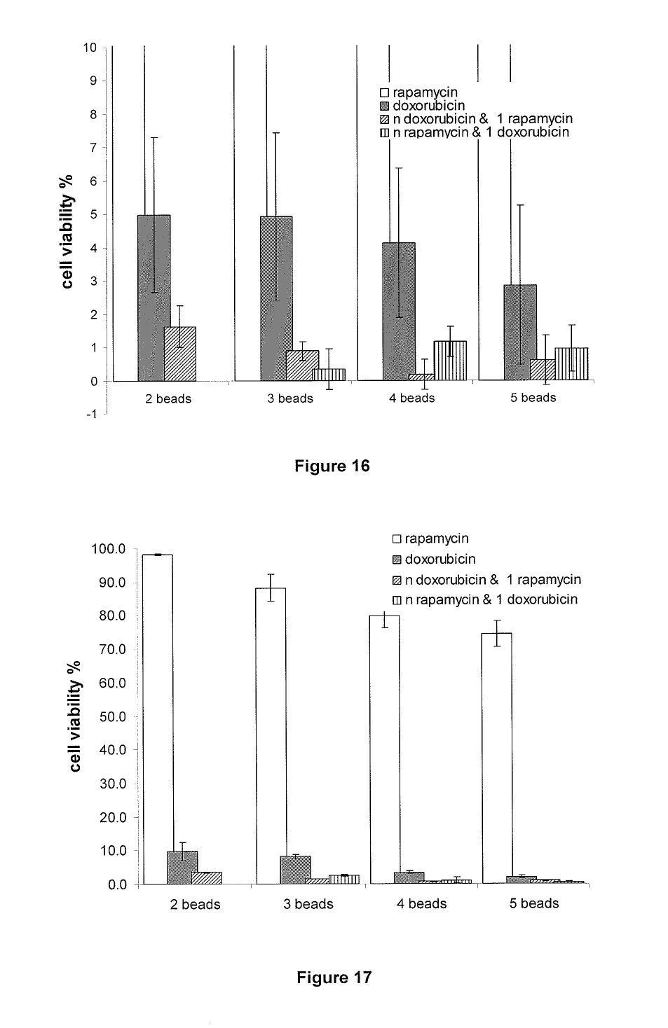

FIG. 16 shows the percentage of viable HepG2 cells for rapamycin, doxorubicin and combination—scale expansion (Error bars: 1SD, n=5), results from LDH assay, as described in Example 7;

FIG. 17 shows the percentage of viable HepG2 cells for rapamycin, doxorubicin and combination (Error bars: 1SEM, n=3), results from MTS assay, as described in Example 7;

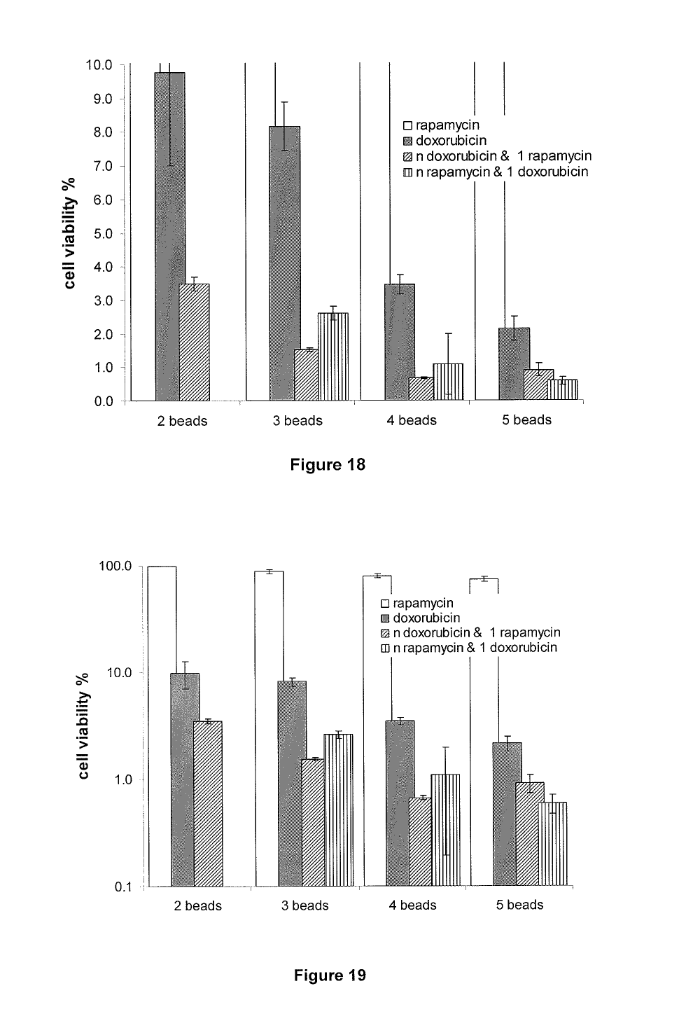

FIG. 18 shows the percentage of viable HepG2 cells for rapamycin, doxorubicin and combination-scale expansion (Error bars: 1SEM, n=3) Results from MTS assay, as described in Example 7;

FIG. 19 shows cell viability % (log scale) of HepG2 cells after 72 hrs exposure to drug loaded microsphere combinations (MTS assay, n=6, ±SD), as described in Example 8;

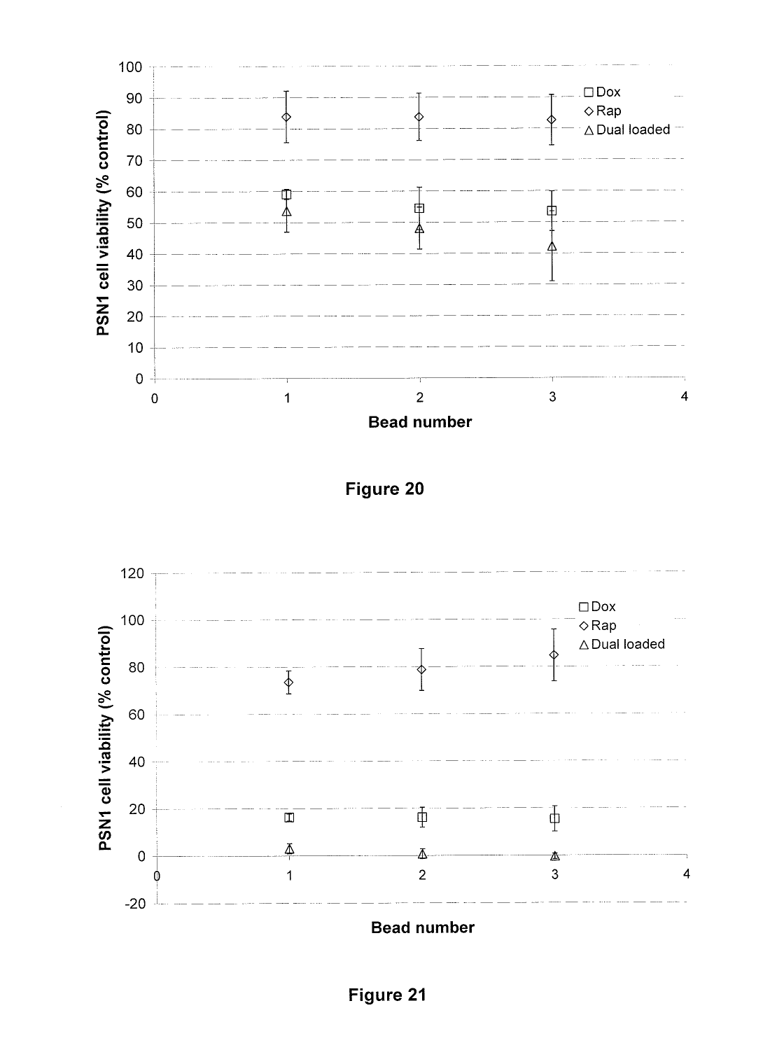

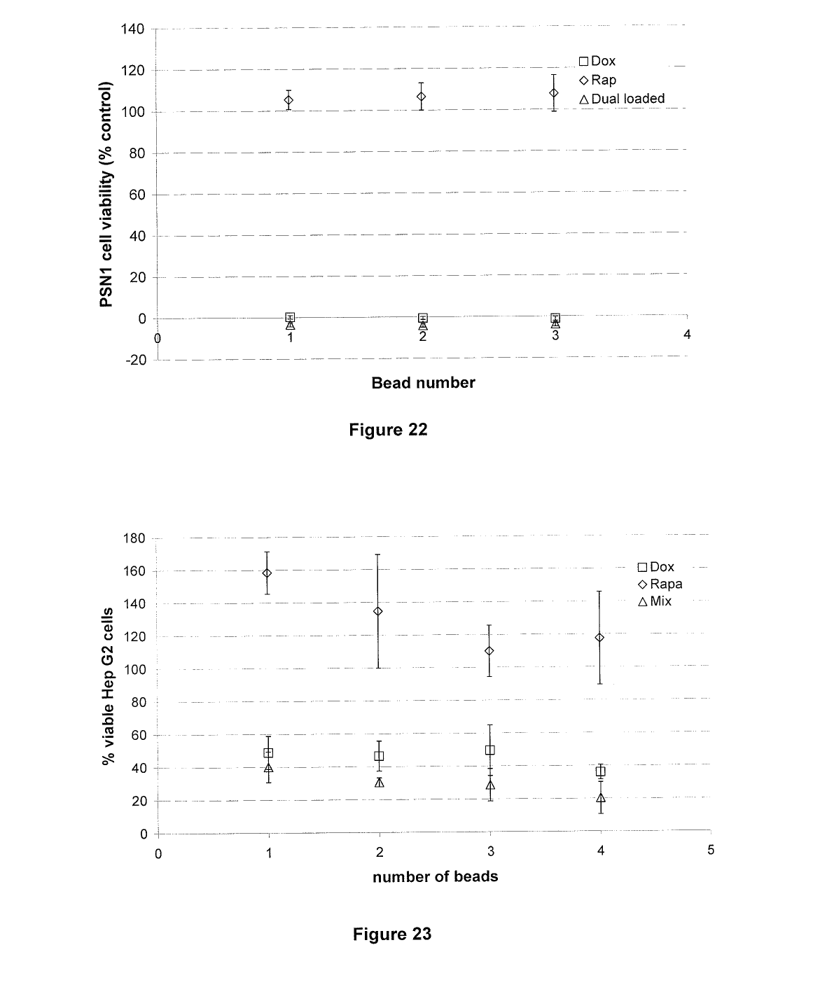

FIGS. 20 to 22 shows the percentage of viable PSN1 cells after 24, 48 and 72 hours (respectively) exposure to mono and dual loaded drug eluting beads (n=7+/−SD) as described in Example 9;

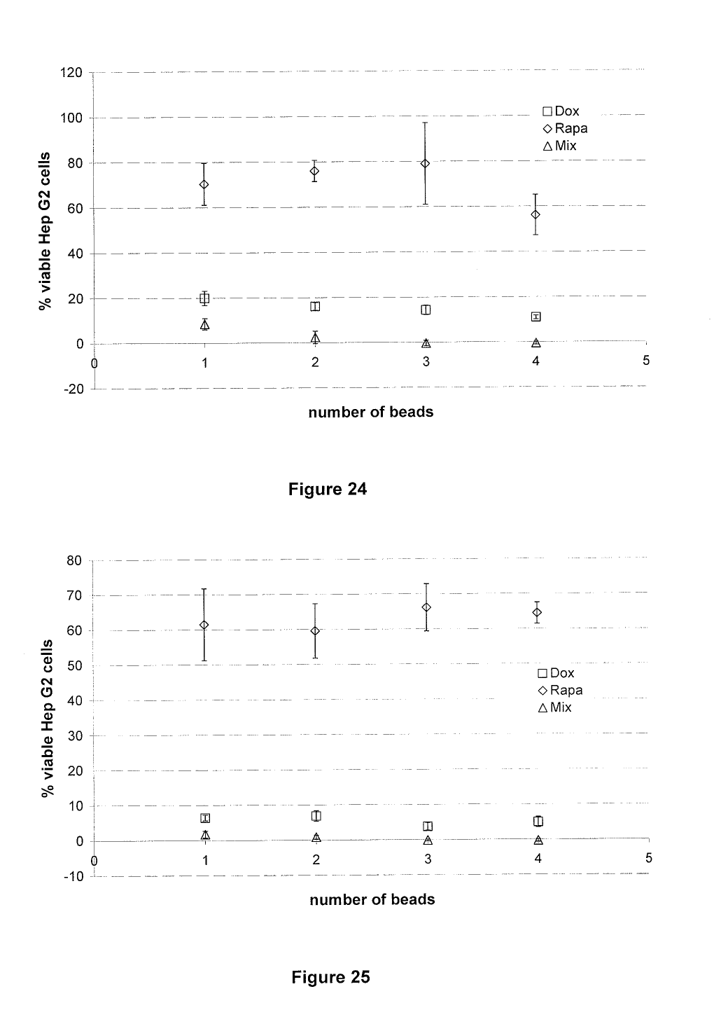

FIGS. 23 to 25 show the percentage of viable HepG2 cells after 24, 48 and 72 hours (respectively) exposure to mono and dual loaded drug eluting beads (n=4+/−SD) as described in Example 9; and

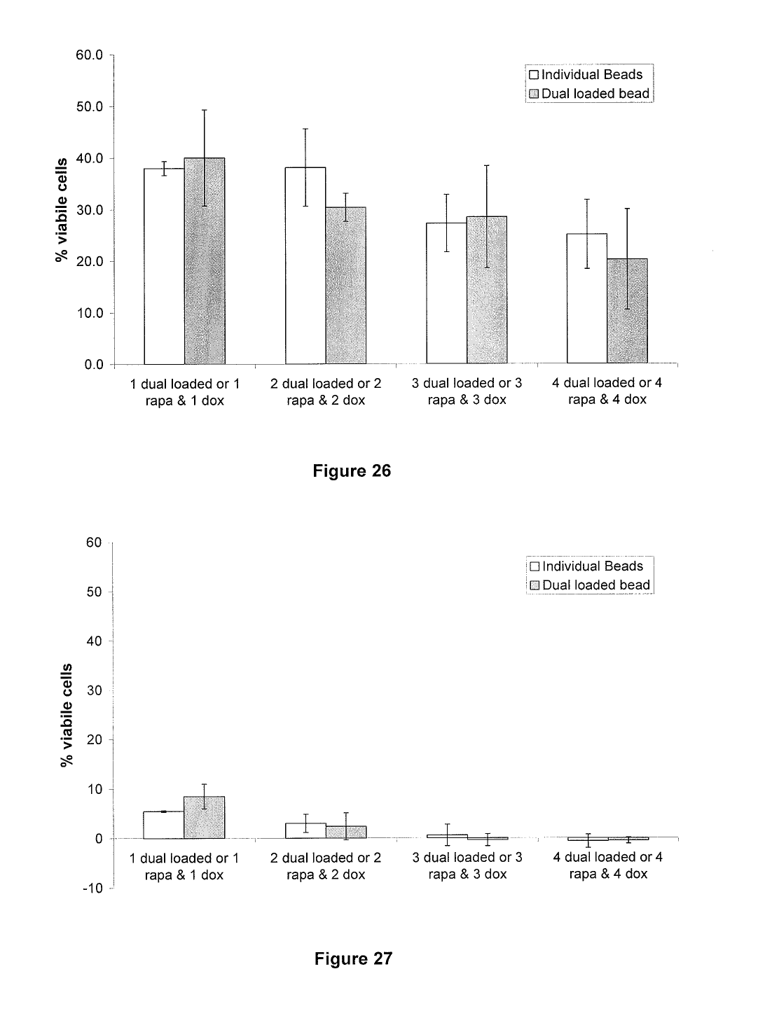

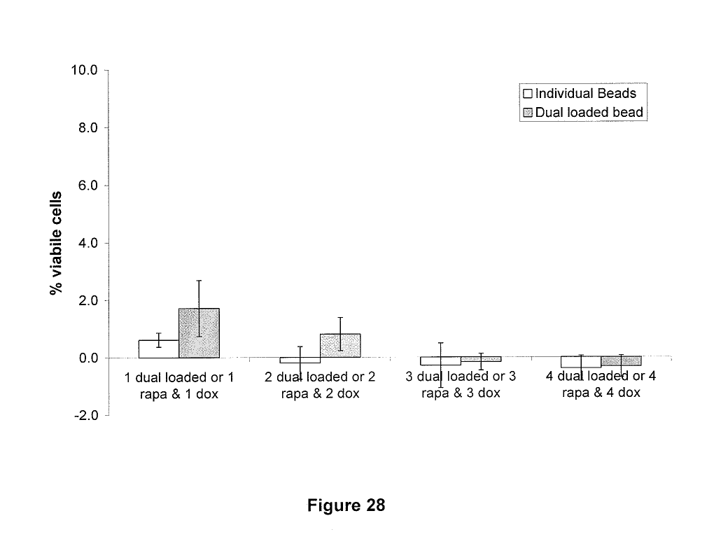

FIGS. 26 to 28 show the percentage of viable HepG2 cells after 24, 48 and 72 hours (respectively) exposure to mono and dual loaded drug eluting beads (n=4+/−SD) as described in Example 10.

### EXAMPLE 1

### Preparation of Drug Eluting Beads

Microspheres are synthesised as described in Reference Example of WO2006027567 to produce low AMPS and high AMPS formulations.

### EXAMPLE 2

### Loading and Elution of Irinotecan and Doxorubicin Combinations

Five samples of 500-700 μm High AMPS beads of Example 1 were loaded with doxorubicin and irinotecan following different approaches:

Sample 1: 3.5 mL of irinotecan solution (Campto, Pfizer, 20 mg.mL⁻¹=70 mg) was loaded into 1 mL of beads for 48 h, to saturate all the sulfonate groups. Then, the depleted solution was removed and the loaded beads were washed with a known volume of deionised water to remove the non-bound drug. The amount of irinotecan loaded into the beads was calculated by depletion, as a difference between the initial and final amounts in the depleted solution and all washings measured by UV at a wavelength of 369 nm. One mL of doxorubicin (Dabur oncology-25 mg.ml⁻¹) was later added to the same beads and the loading profile determined.
Sample 2: Similar to sample 1, but 2.8 mL of doxorubicin (25 mg.ml⁻¹=70 mg) solution was added to the beads instead of the irinotecan solution in the first step, to saturate all the sulfonate groups. All solutions were measured by UV at a wavelength of 483 nm wavelength and the amount of drug loaded into the beads determined by depletion. After the beads were loaded and washed with deionised water, in a second step, 1.25 ml of irinotecan solution (20 mg.mL⁻¹=25 mg) was added to the same beads and the loading profile determined.
Sample 3: 1.25 mL of irinotecan solution (20 mg.ml⁻¹=25 mg) was loaded into 1 ml of beads and after 1 h, 1 ml of doxorubicin (25 mg.ml⁻¹) was added to the same beads. No intermediate washes were performed in this or the following samples.
Sample 4: 1 ml of doxorubicin (25 mg.ml⁻¹) was loaded into 1 ml of beads and after 1 h 1.25 mL of irinotecan solution (20 mg.ml⁻¹=25 mg) was added to the same beads.
Sample 5: 1.25 ml of irinotecan solution (20 mg.ml⁻¹=25 mg) was mixed with 1 ml of doxorubicin (25 mg.ml⁻¹) and then added to 1 ml of beads.

To determine the loading profiles of all samples, an aliquot of 50 μl was removed from each vial at time 5, 15, 30, 45 min after the addition of each drug, diluted as necessary and read by UV at 483 and 369 nm. The amount loaded at each time point was determined by depletion using the corresponding standard curves.

After loading with both drugs, all samples were sized in their loading solutions using the Colorview III camcorder and pictures were taken. For that, at least 100 beads were measured. These data were normalised and the corresponding histograms generated.

After being sized, all samples were eluted. Here, the remaining loading solutions were removed using a Pasteur pipette and the loaded beads were transferred to brown flasks containing 200 ml of PBS (Inverclyde Biologicals). At time 5, 15, 30, 45, 60, 90 min, 2 h, 4 h and 24 h, an aliquot of elution medium (4 ml) was removed and replaced with 4 mL of fresh PBS to maintain the volume. All samples were read by UV at 483 and 369 nm wavelengths, using the corresponding standard curves to calculate the amount eluted at each time point.

### Calculations:

To calculate the loading and elution of both drugs from the same beads the following assumptions were made:

Absorbance at 483 nm=Absorbance of doxorubicin at 483 nm.

It is observed in the UV scan of irinotecan solution that this drug does not absorb at this wavelength.

Absorbance 369 nm=Absorbance of doxorubicin at 369 nm+Absorbance of irinotecan at 369 nm.

The doxorubicin scan showed some absorbance at 369 nm wavelength and it is considered here that there is not interaction between the absorbance of doxorubicin and irinotecan when measured at the same time. The scan from the mixture does not show any notable interaction between the absorbance spectra of both drugs.

These two assumptions were made for all calculations. All samples were read at both 483 nm and 369 nm wavelengths. Using the absorbance and the standard curve of doxorubicin at 483 nm, the concentration of doxorubicin in each sample was calculated and using these concentrations and the standard curve of doxorubicin at 369 nm, the absorbance of doxorubicin at this wavelength was calculated. Then, subtracting this value from the total absorbance at 369 nm, the absorbance at 369 nm and hence the concentration of irinotecan was calculated.

Loading of Irinotecan and Doxorubicin into the Same Beads

The objective of these experiments was to assess and compare the loading and elution behaviour of different combinations of doxorubicin and irinotecan in order to determine their mechanism of interaction with the beads and evaluate possible interactions between drugs. If both drugs could be successfully incorporated in the same beads it would be simple to achieve the local delivery of both antitumour drugs simultaneously if required.

Doxorubicin and irinotecan were loaded following the different approaches detailed above and results are presented as follows:

As seen in FIG. 1, if irinotecan is loaded at maximum bound capacity first and then doxorubicin is added to the system (Sample 1), some irinotecan (˜17%) is displaced, but the majority of drug is retained by the beads. If doxorubicin is loaded at maximum bound capacity first and then irinotecan is added to the system (Sample 2), a smaller percentage of doxorubicin (<8%) is displaced by the irinotecan. These data agree with the fact that the K_{b} values for doxorubicin are slightly higher than those calculated for irinotecan (Gonzalez et al 2006) and doxorubicin releases much slower and to a lower extent than irinotecan in the same volume of solution. In both cases, however, the amount of drug displaced is small, indicating both interactions are stable after addition of the second drug. This could be explained as a consequence of the small volumes of solution used, easily saturated, in the loading process and the initially-bound bulky drug molecule sterically hindering the approach of the second drug to the binding sites, a phenomenon that would not be experienced by small ions, such as Na⁺ or K⁺. These two factors will contribute to the small percentage of drug displaced by the second drug added.

Sample 3 and 4 in FIG. 2 show that if the beads are first loaded with 25 mg of one drug and then 25 mg of the other drug is added, very little of the bound drug (<5%) is displaced on addition of the second drug, independent of the order. These results are expected, as the maximum loading capacity of the beads is ˜40 mg and 52 mg for doxorubicin and irinotecan respectively and the binding sites of the beads are not saturated when the second drug is added (initially, 25 mg of drug are added to the beads and then 25 mg of the other drug is added).

Sample 5 on FIG. 2 shows that if both drugs are loaded at the same time, the loading rate and the total amount loaded is slightly higher for doxorubicin than for irinotecan (94% of the total doxorubicin added (25 mg) is loaded whereas 91% of the total irinotecan added (25 mg) is loaded after 2 h). This small difference could be explained as a result of their K_{b} values, where doxorubicin K_{b} value (5.58*10⁸−1.02*10⁹) is slightly higher than for irinotecan (8.73*10⁷−3.62*10⁸).

Sizing of Irinotecan and Doxorubicin Loaded into the Same Beads

No notable differences were detected between beads loaded with both drugs by the different approaches (FIG. 3).

Elution of Irinotecan and Doxorubicin Loaded into the Same Beads

FIG. 4 shows that all samples follow similar elution trends and doxorubicin is released very slowly (<25% in 24 h) compared to irinotecan (>99% mg in <4 h) when eluted into 200 ml of PBS.

In samples 1 and 2, where one drug was loaded up to saturation level before the other drug was added, the second drug to be loaded was released faster than in sample 3 and 4, where both drugs were loaded into the beads at the same time below saturation level. This could be explained by the partially bound nature of the second drug to be loaded, caused by all the sulfonate groups being saturated (sample 1 and 2). A small amount of the drug loaded second would be bound to the beads by displacing the first, mostly bound drug. Most of the loaded drug would be free (unbound) inside the beads and would elute faster than the bound drug.

In conclusion, when doxorubicin and irinotecan are loaded at the same time or consecutively into the beads, no change in the release behaviour of the other drug is observed. The presence of an additional drug does not interfere the loading or modify the release behaviour of the other drug. Each drug elutes as expected from the individual drug elution profiles, generated for each drug alone.

UV profiles of the eluted solutions were performed and compared to an unloaded mixture of doxorubicin and irinotecan. The shape of the peaks and the maximum values did not change, confirming that no apparent interaction or modification of either drug occurs inside the beads.

These experiments demonstrate that is possible load and elute both drugs at the same time from the same beads. Sustained release of the two drugs is expected from this product. The combination of these two anti-tumoural drugs is expected to achieve higher therapeutic effect than the treatment with a high-dose single drug and to decrease the adverse effects of both drugs. This potential product will be evaluated in vivo to determine their effect as synergistic, additive or antagonistic. This effect will depend on the molecular ratios of individual chemotherapy agents and schedules of administration (Aschele et al. 1998). In vivo studies will be carried out to evaluate different doses of the two agents in order to determine the best ratio. These experiments revealed that both drugs could be successfully loaded and eluted within the same beads without alter the release profiles of the drugs individually and suggest the possibility of loading more than one cationic drug in the beads.

### EXAMPLE 3

### Combination of Ibuprofen and Doxorubicin in the Same Beads

Ibuprofen and doxorubicin solutions were loaded into low AMPS beads simultaneously. In order to achieve this, one sample of 1 ml of 500-700 μm beads was loaded with ibuprofen (according to examples in WO2004073688). The excess packaging solution was removed using a Pasteur pipette and 2 ml of doxorubicin solution (5 mg.ml⁻¹) were added. After 24 h, a 50 μl aliquot was removed from the depleted solution and measured by UV at 263 nm and 483 nm. The amount of doxorubicin loaded into beads was calculated by depletion, as a difference between the initial and final amounts in the depleted solution (calculations explained below).

After loading with both drugs, the depleted loading solution was removed and the loaded beads were transferred to brown flasks containing 200 mL of PBS. At time 5, 45, 90 min, 3, 5 and 24 h, 2 ml of elution medium was removed and replaced with 2 ml of fresh PBS to maintain the volume. All samples were read by UV at 263 and 483 nm and the amount of each drug eluted at each time point was calculated using the corresponding standard curves.

### Calculations:

To calculate the loading and elution of both drugs from the same beads the following assumptions were made:

Absorbance at 483 nm=Absorbance of doxorubicin at 483 nm.

The UV scans of ibuprofen and doxorubicin solution showed that only doxorubicin absorbs at this wavelength.

Absorbance 263 nm=Absorbance of doxorubicin at 263 nm+Absorbance of ibuprofen at 263 nm.

The doxorubicin UV scan showed some absorbance at 263 nm wavelength. The assumption is made that there is no interaction between the absorbance of doxorubicin and ibuprofen when measured at the same time.

These two assumptions were made for all calculations. All samples were read at both wavelengths (263 nm and 483 nm). Using the absorbance at 483 nm the concentration of doxorubicin in each sample was calculated, then using these concentrations and the standard curve of doxorubicin at 263 nm, the absorbance of doxorubicin at 263 nm was calculated. By subtracting this value from the total absorbance at 263 nm, the concentration of ibuprofen, was calculated using the ibuprofen standard curve.

### Loading and Release of Doxorubicin and Ibuprofen Combinations in Beads

In the first set of experiments, doxorubicin and ibuprofen were combined into the same beads and their release behaviour was evaluated.

Initially, lyophilised beads as described in WO2004073688 were loaded with ibuprofen/ethanol solution by solvent evaporation and then rehydrated with water and autoclaved for 15 min at 121° C. The rehydrated ibuprofen loaded beads were then loaded with doxorubicin by immersion into doxorubicin solution (5 mg.ml⁻¹) for 24 h. In this experiment, a maximum amount of 8.3 mg of doxorubicin was loaded into the beads calculated by depletion of the concentration of the loading solution. The low AMPS beads have a maximum capacity for doxorubicin of ˜14 mg.ml⁻¹ when loaded with this drug alone. This maximum amount is limited by the number of binding sites available in the beads. If a greater amount of drug were to be added to the sample, it would not be loaded as all the sulfonate groups within the beads would have been saturated.

The release of both drugs from the same beads was monitored by UV spectrophotometry at the same time, the elution profile for both drugs released is presented in FIG. 5. Both drugs had similar release profiles to those exhibited for beads loaded with only one of the drugs and no interaction was observed between the two drugs. This experiment showed that both drugs can be loaded and released into the same bead, offering a platform for sustained released of two drugs simultaneously. This could be beneficial as a drug delivery device by adding the therapeutic effects of both drugs by administration of one product. This therapy could reduce the requirements of analgesic drugs following the procedures, and the dose of the more toxic drug if synergistic antitumoral results by the combination of both drugs are demonstrated.

### EXAMPLE 4

### Combination of Doxorubicin and Rapamycin in the Same Beads

Loading Methods: Two Loading Methods were Tested as Shown in FIG. 6).

### Method 1

Unloaded high AMPS bead slurry 1 ml was loaded with doxorubicin solution (5 ml of 10.07 mg/ml, target loading 50 mg/ml Bead) overnight. The doxorubicin-loaded beads were washed with 1 ml DMSO 5 times to remove extra water, and 1 ml 60 mg/mL rapamycin DMSO solution was mixed with the doxorubicin beads for about 30 min. Subsequently, following removal of extra rapamycin DMSO solution, the beads were washed with 5 ml saline until no white drug particles were observed.

### Method 2

1 ml Bead slurry was washed with 1 mL DMSO 5 times and mixed with 1 ml 60 mg/ml rapamycin DMSO solution for 30 min. The loaded beads were then washed with 5 ml saline until no further white drug particles were observed. 0.5 ml of rapamycin-loaded bead slurry was roller-mixed with 2.5 ml 10.07 mg/ml doxorubicin solution overnight, target doxorubicin loading is 50 mg/ml.

Doxorubicin loading test: The doxorubicin loading was measured by depletion method in either water or DMSO solution. The loading solution was taken and diluted 10 times and measured by UV at 483 nm to determine the concentration of doxorubicin residue in solution.
Rapamycin loading test: For the beads with rapamycin alone, 0.5 ml rapamycin-loaded beads (from 1^{st} step, Method 1) were extracted by 1 ml DMSO 5 times, and the solution were accumulated in a 10 ml volumetric flask. The concentration of rapamycin was determined by UV at 279 nm.

For the beads with doxorubicin and rapamycin combination, 0.26 ml of drug-loaded beads (from 2^{nd} step, Method 2) were extracted with 1 ml DMSO 4 times, and the extraction was collected in a 5 ml volumetric flask and DMSO was added to top the solution to the mark. The concentration of rapamycin was determined by subtraction of the absorbance of doxorubicin at 279 nm from the total absorbance at 279 nm, in which the doxorubicin absorbance at 279 nm was calculated from that at 483 nm according to the scan of concentration-known solutions.

Doxorubicin loss determination: The doxorubicin loss during DMSO washing of loaded beads before further loading of rapamycin (Method 2) was determined by measuring the accumulated DMSO washing solution in a volumetric flask at 483 nm.
Elution of drug-loaded beads: The elution of drug-loaded beads were carried out by mixing the beads with 200 ml PBS (Inverclyde, Bellshill, UK) and roller-mixed on a roller mixer. At certain time point, the solution (100 ml or 150 ml) was taken out for concentration determination by UV and same volume of fresh PBS was added to keep a sink condition. The determination rapamycin concentration followed the same principle as that in the section of Rapamycin Loading Test.

Image analysis and sizing: The loaded beads were placed under an Olympus microscope, about 200 beads were counted for size distribution study by software (AnalySIS 5.0 Soft Image System GmbH), and photos were taken under different light/contrast background for morphology study.

Rapamycin and doxorubicin standard curve: The maximum absorbance of rapamycin is at wavelength 279 nm, which is overlapped with doxorubicin absorbance. At 483 nm, the wavelength of maximum absorbance of doxorubicin, rapamycin has no absorption. Therefore, the rapamycin concentration in a bi-component system (doxorubicin and rapamycin) could be determined by first measurement of standard solutions of doxorubicin at both wavelength, then subtraction of the absorbance of doxorubicin from the absorbance of mixture. Standard curves are created of doxorubicin at 279 and 483 nm in water and DMSO.
The concentration of rapamycin after subtraction of doxorubicin absorbance can be determined by the relationship given underneath at 279 nm:

Absorbance=56.18×Concentration (mg/ml)[in DMSO]

Absorbance=44.64×Concentration (mg/ml)[in water]

### Comparison of Drug Loading in the Two Loading Procedures

In the 1^{st} step of Method 1, rapamycin was loaded at 28.48 mg/ml, the loading efficiency was about 47.5% (Table 1). Compared to the rapamycin loading in the 2^{nd} step of Method 2, 37.22 mg/ml rapamycin was loaded, the loading efficiency being 62.0%. This significantly high loading efficiency was consistent with the observation that the doxorubicin-loaded beads swelled larger and most of the rapamycin loading solution was sucked into the beads. From a mechanism point of view, the doxorubicin-loaded beads have smaller size, and tend to swell larger in polar solvents like water and DMSO due to the reduced salt effect which was observed in doxorubicin loading solution or saline.

For the beads loaded with rapamycin (28.48 mg/ml), the doxorubicin loading capability was found to be 46.02 mg/ml, similar to that of beads without rapamycin. During doxorubicin loading, no rapamycin leakage was observed. This experiment suggests that rapamycin preloading does not affect the later doxorubicin loading, which means that the two drugs occupy different sites within the beads. Doxorubicin molecules tend to bind to the site of the sulfonate groups by ionic interaction; however, rapamycin stays within the interstitial space within the hydrogel by expelling water out and forming small drug crystals.

TABLE 1
Rapamycin and doxorubicin loading by two methods^{a}

| | Doxorubicin | Rapamycin |
|---|---|---|
| | loading | loading |
| Sample list | (mg/ml^{b}) | (mg/ml^{b}) |
| Rapamycin loading in 1^{st} step, | — | 28.48 |
| Method 1 | | |
| Doxorubicin loading in 1^{st} step, | 45.88 | — |
| Method 2 | | |
| Doxorubicin loading in 2^{nd} step, | 46.02 | 28.48^{c} |
| Method 1 | | |
| Rapamycin loading in 2^{nd} step, | 39.17^{d} | 37.22 |
| Method 2 | | |

^{a}The data shown in the table are average values of two replicates.
^{b}All the drug loading data were normalised to the volume of beads without drug loading.
^{c}No rapamycin loss was observed during doxorubicin loading, therefore the same data as that in 1^{st} step was used.
^{d}The doxorubicin loss during DMSO washing in preloading of rapamycin was 6.71 mg/ml. The further doxorubicin loss during rapamycin loading was ignored.

In the Method 2, the washing of doxorubicin-loaded beads with DMSO before rapamycin loading caused doxorubicin loss of about 6.71 mg/ml, although this method had a higher rapamycin loading. The final doxorubicin to rapamycin ratio was 1.62 for beads from Method 1, and 1.05 for beads from Method 2.

From an operational point of view, Method 1 is a relatively clean procedure and only produces rapamycin waste solution. Method 2 produces both doxorubicin-DMSO and rapamycin waste solution. Doxorubicin loading in Method 1 is almost completed, and Method 1 is also relatively easier to process. In spite of high loading of rapamycin in Method 2, Method 1 is recommended.

### Image Analysis and Size Distribution of Drug-Loaded Beads

Photographs are taken after rapamycin and rapamycin-doxorubicin loading by Method 1. It appears that these beads retain their smooth surface and spherical shape well after drug loading. Some small particles of rapamycin were observed, which are the residues from the 1^{st} step. The doxorubicin-loaded beads experienced a slight lost of their spherical shape. After additional loading with rapamycin, the shape recovered well and their surfaces were smooth. If compared to the final beads from Method 1, the beads from Method 2 have shallow red colour, which may be due to doxorubicin loss after DMSO washing, or due to later loaded rapamycin coverage.

The size characterisation of DC Bead and drug-loaded beads were listed in Table 2, and the size distribution was shown in FIG. 7. From Table 2, the rapamycin-loaded beads in Method 1 have a slight increased sizes compared to unloaded beads. Once loaded with doxorubicin, the mean size decreased by about 100 μm. In Method 2, the doxorubicin-loaded beads show the smallest mean size. After loading with rapamycin, the size increased to about 479 μm, close to the final bead size from Method 1. Meanwhile, the standard deviation data in Table 2 indicate that the range of size distribution has almost no change during drug loading. Therefore, the two methods give final beads having almost the same size range (FIG. 7).

TABLE 2
Size characterisation of Unloaded and drug-loaded beads

| | | Min- | | |
|---|---|---|---|---|
| | Maximum | imum | Mean | Standard |
| | size | size | size | deviation |
| Sample list | (μm) | (μm) | (μm) | (μm) |
| Unloaded Beads | 645 | 451 | 546 | 46 |
| After rapamycin loading in 1^{st} | 662 | 471 | 560 | 48 |
| step, Method 1 | | | | |
| After doxorubicin loading in 1^{st} | 518 | 365 | 444 | 39 |
| step, Method 2 | | | | |
| After doxorubicin loading in 2^{nd} | 532 | 360 | 466 | 36 |
| step, Method 1 | | | | |
| After rapamycin loading in 2^{nd} | 562 | 402 | 479 | 40 |
| step, Method 2 | | | | |

Drug Elution from Loaded Beads

FIG. 8 shows the elution data of doxorubicin and rapamycin from loaded beads into PBS. In this experiment, the elution medium was refreshed after a certain time to mimic sink conditions. From FIGS. 8 (B) and (D), the drug elution profiles from the beads made by the different methods are very similar: rapamycin released about 33% after 30 hr elution, and doxorubicin released about 41 to 46%.

Comparing FIGS. 8 (A) and (C), the doxorubicin from beads made by Method 1 eluted slightly faster than the beads made by Method 2. For rapamycin elution, the reverse phenomenon was observed. This may be due to the later loaded drug covered or blocked the pathway of the first drug, which was also used for the first drug elution.

### EXAMPLE 5

### Combination of Irinotecan and Ibuprofen in the Same Bead

Drug loading: The drug loading procedure is shown in FIG. 9, in which the unloaded high AMPS beads were washed with DMSO to remove the water trapped within the hydrogel. A mixture of ibuprofen solubilised in DMSO and castor oil as crystallisation inhibitor were subsequently mixed with the bead slurry, and kept for about 10 minutes. Following the removal of the loading solution, saline was used to wash the loaded beads until no drug particles nor large oil droplets were observed. The irinotecan loading was achieved by simply mixing the ibuprofen-loaded beads with irinotecan solution (10 mg/mL) by roller-mixing overnight. The final loaded beads were kept in the water solution as product.

The sample matrix of ibuprofen-irinotecan beads was listed in Table 3.

TABLE 3
Sample matrix of ibuprofen-irinotecan loading

| | | | | Irinotecan loading |
|---|---|---|---|---|
| | Composition of ibuprofen loading | | | solution |
| | solution | | | (10.07 mg/mL) |
| | Ibuprofen | DMSO | Castor oil | Volume of Irinotecan |
| Sample | (g) | (ml) | (g) | solution (ml) |
| Ex 5-1 | 0.8 | 5 | 5 | 4 |
| Ex 5-2 | 0.4 | 5 | 5 | 4 |
| Ex 5-3 | 0.2 | 5 | 5 | 4 |
| Ex 5-4 | 0.8 | 2.5 | 7.5 | 4 |

### Determination of Drug Content in Beads:

1). Ibuprofen. The content of loaded ibuprofen was determined by an HPLC method. The slurry of drug-loaded beads with known volume was extracted with DMSO (1 ml) for five to six times. The collected DMSO solution was topped to the mark in a 10 ml volumetric flask, and the concentration of ibuprofen was measured by HPLC.
2). Irinotecan. The content of irinotecan in loaded beads was measured by depletion method. 1-1.1 ml of ibuprofen-loaded beads were roller-mixed overnight with 4 mL 10 mg/ml irinotecan solution to target the loading at 40 mg/ml beads. The loading solution was diluted with water and measured by UV at 369 nm. Compared to the standard curve, the concentration of irinotecan left in loading solution can be calculated.

### Elution of Drug-Loaded Beads:

The elution of ibuprofen-irinotecan-loaded beads was carried out by mixing 0.24 ml beads with 200 ml PBS (Inverclyde, Bellshill, UK) and roller-mixed on a roller mixer. At certain time point, the solution (5 ml) was taken out for concentration determination by UV and same volume of fresh PBS was added to keep a sink condition.

### Image Analysis and Sizing:

The loaded beads were placed under an Olympus microscope, about 200 beads were counted for size distribution study by software (AnalySIS 5.0 Soft Image System GmbH), and photos were taken under different light/contrast background for morphology study.

### Determination of Concentration of Combination of Ibuprofen and Irinotecan

To determinate the concentration of each component in a combined drug solution, the standard curves for absorption of predetermined concentrations of ibuprofen and irinotecan at 263, 272 and 369 nm in water were measured. The principle is to determine the concentration of irinotecan from the absorbance at 369 nm first, where ibuprofen has no absorption. Then the concentration of ibuprofen could be calculated by subtracting the irinotecan absorbance at 272 nm from the spectrum of drug mixture. Using 272 nm instead of 263 nm commonly used is to reduce the possible interference of DMSO residue in drug loading. However, it was found that the ibuprofen absorbance is much lower compared to the irinotecan due to the low water-solubility of ibuprofen, and the ibuprofen peak was fully covered by irinotecan. Therefore the way using UV to determine ibuprofen concentration in ibuprofen-irinotecan mixture is not accurate. In this study, the irinotecan concentration was measured by UV, and ibuprofen concentration was measured by HPLC.

Loading Ibuprofen and Irinotecan into Beads

All the samples loaded with ibuprofen in the first step retain their spherical shape and no ibuprofen crystals were seen to form. After irinotecan loading, Example 5-1 with high ibuprofen loading had a large amount of ibuprofen crystals appear on the bead surface. In comparison, the Example 5-4 with the same amount of ibuprofen has no crystals observed, which is due to the effect of the high content of castor oil crystallisation inhibitor existing within beads. Therefore, the castor oil acts as an excipient to prevent the formation of ibuprofen crystals by inhibiting crystal formation.

Table 4 lists the drug loading in each sample. The irinotecan loading in all the samples is close to 100%, the maximum irinotecan loading capacity for unloaded beads being close to 50-60 mg/ml, and below that the loading efficiency is above 98%.

TABLE 4
Drug loading in Unloaded Beads

| | | | Target | Actual |
|---|---|---|---|---|
| | | | irinotecan | irinotecan |
| | Target ibuprofen | Actual ibuprofen | loading | loading |
| Sample | loading* (mg/ml) | loading (mg/ml) | (mg/ml) | (mg/ml) |
| Ex 5-1 | 40 | 14.34** | 40.28 | 40.28 |
| Ex 5-2 | 20 | 10.41 | 40.28 | 40.28 |
| Ex 5-3 | 10 | 5.79 | 40.28 | 40.28 |
| Ex 5-4 | 40 | 11.68 | 40.28 | 40.28 |

*The data were estimated according to the total volume of DMSO and castor oil treated as about 10 ml.
**Including the ibuprofen crystal on bead surface.

The ibuprofen loading is relatively low, and the estimated loading efficiency is between 29% to 58%. The lower target of loading of ibuprofen gives a relatively higher loading efficiency. The reason of the relatively low ibuprofen loading is due to the bead size shrinking when the beads contact with castor oil rich solution. Hence, the drug loading decreased with the smaller volume of beads.

### Size Distribution of Drug-Loaded Beads

The size distribution of drug-loaded beads is shown in FIG. 10. The corresponding parameters are listed in Table 5. The ibuprofen-loaded beads almost keep the same size as the DC Bead without drug loading, in the range from 500 to 700 μm. However, the size decreased significantly with irinotecan loading due to the increased hydrophobicity of the drug-loaded beads, the average size decrease is about 140 to 150 μm. It is noticed that the beads with variance of ibuprofen loading have almost the same mean size and size distribution.

TABLE 5
Comparison of size distribution for drug loaded beads

| | | Sizeₘₐₓ | Sizeₘᵢₙ | Sizeₘₑₐₙ | SD |
|---|---|---|---|---|---|
| Sample | Drug loaded | (μm) | (μm) | (μm) | (μm) |
| 849BB/20-1 | IBU* | 690 | 476 | 589 | 53.5 |
| 849BB/20-1 | IBU-IRI** | 549 | 340 | 448 | 41.7 |
| 849BB/20-2 | IBU | 688 | 470 | 592 | 47.4 |
| 849BB/20-2 | IBU-IRI | 510 | 354 | 432 | 33.6 |
| 849BB/20-3 | IBU | 670 | 474 | 585 | 56.0 |
| 849BB/20-3 | IBU-IRI | 532 | 371 | 444 | 43.7 |
| 849BB/20-4 | IBU-IRI | 527 | 362 | 446 | 36.1 |

*IBU: ibuprofen
**IRI: irinotecan

### Morphology of Drug-Loaded Beads

A micrograph of ibuprofen-loaded beads of Example 5.1, demonstrates that the structure of the loaded beads consist of a large core area with ibuprofen and castor oil, and a layer on the bead surface free of drug and oil. Castor oil trapped inside the beads provides the hydrophobic phase to retain ibuprofen and to prevent the formation of ibuprofen crystals. The other Examples 5-2, 5.3 and 5.4 with ibuprofen in the first loading step have the same structure shown here. In addition, some small oil droplets were observed among the beads, which were the residues of mixture of castor oil and ibuprofen in solution.

When irinotecan was loaded by roller-mixing overnight, ibuprofen crystals were observed on the surface of the beads in Example 5.1, and bead aggregation occurred as the result of crystal formation. Crystallisation was not found in other samples. The phenomenon is due to the higher ibuprofen content in the beads, and not enough castor oil to prevent crystal formation, when irinotecan was loaded. Although the irinotecan interacts with the AMPS group in the beads, it still is hydrophobic and may tend to partially dissolve in castor oil, which could reduce the solubility of ibuprofen. In the product of Example 5.4, with a higher level of castor oil and high loading of ibuprofen, after irinotecan loading, no ibuprofen crystals were observed.

### Elution of Drug-Loaded Beads

FIGS. 11 and 12 show the elution profile of irinotecan and ibuprofen from beads into PBS at 25° C. In FIG. 11, the elution rate of irinotecan under different castor oil content and ibuprofen loading are almost the same. The irinotecan elution rate is also comparable to that for irinotecan alone in which almost 100% of the drug was eluted after 5 hours. This indicates that castor oil and ibuprofen loading have no effect on irinotecan elution, and there is no interaction between ibuprofen and irinotecan. In FIG. 12, the ibuprofen elution is completed after 1 to 2 hr in PBS. Compared to the ibuprofen elution from single drug-loaded beads, the elution again is similar indicating no drug-drug interaction is occurring.

### EXAMPLE 6

### In vitro Cytotoxicity Evaluation of Doxorubicin Loaded Beads using Human HCC Cells (Reference)

Doxorubicin beads (500-700 μm) were prepared according to the methods outlined in Example 2 of WO04071495. The elution of doxorubicin loaded beads in cell culture media with a human hepatocellular liver carcinoma (HepG2) cell line was monitored. A Promega lactate dehydrogenase (LDH) assay was used to assess the cytotoxicity effects on the HepG2 cell line at 24, 48 and 72 hour time points.

### Assay Preparation

Using aseptic conditions, three sterile flat bottomed 96 well plates (Cellstar®, 655180) were seeded with 20,000 HepG2 cells in 200 μl media and left in an incubator (37° C., 5% CO₂) for 20 hours. After incubation, the media was carefully aspirated from all of the wells and 200 μl of PRF media was added to the wells. One, three and ten pre-loaded doxorubicin beads (37.5 mg per ml of beads) were counted under aseptic conditions into 200 μl phenol red free cell culture media in a 96 well plate.

### Doxorubicin Elution Assay

The plate was incubated at 37° C., 5% CO₂. At 24, 48 and 72 hour time points, 100 μl of each well were scanned at 490 nm (Biotek, ELX800). The elution was calculated by comparing the absorbance to a standard calibration curve.

At longer time periods and higher bead numbers more doxorubicin was released into the cell culture media (FIG. 13). The elution profile is different for different bead numbers; the plateau for 10 beads at later time period may be due to saturation of the cell culture media with doxorubicin.

### Cell Viability Assay

This assay measures the amount of LDH released from the remaining viable cells after the dead cell debris have been washed away. The LDH is released after the viable cell membranes have been compromised by freezing the cells.

At each time point, the media and beads was carefully aspirated from the original plate and 200 μl sterile PBS was added and removed three times to wash the cells of any residual doxorubicin and left over cell debris. 200 μl PRF media was added before the plate was subjected to −80° C. for one hour; this intentionally compromised all of the cell membranes, releasing all lactate dehydrogenase enzyme from the remaining live cells. Upon removal from the freezer the plate was promptly placed in an incubator under standard conditions (37° C., 5% CO₂) for thirty minutes. 50 μl of the supernatant was removed from all the wells into respective fresh wells, followed by 50 μl of the LDH assay substrate mix (Promega). The original plate with cells was kept for further analysis. The fresh plate was left a room temperature in the dark. After 30 minutes 50 μl of stop solution (acetic acid (1 M)) was added to terminate the reaction. The plate was then read using a plate reader (Biotek) at 490 nm, if the reading was too high (>3) the solution was diluted by taking 75 μl supernatant and 75 μl PRF media. The percentage of compromised cells was calculated.

The number of viable cells decreases as the exposure to and number of pre-loaded doxorubicin DC beads increases (FIG. 14).

### EXAMPLE 7

### Cytotoxicity of Combinations of Doxorubicin Beads and Rapamycin Beads and In-Vitro

A LDH assay was used to assess the cytotoxicity effects on the HepG2 cell line at 24, 48 and 72 hour time points with different rapamycin/doxorubicin bead combinations.

The rapamycin beads (500-700 μm) were loaded and analysed described in PCT/EP2007/50690. The resulting loading was 21.4 mg/ml.

The doxorubicin beads (500-700 μm) were loaded with 37.5 mg per ml of beads as described in WO04071495.

### Assay Preparation

Using aseptic conditions, three sterile flat bottomed 96 well plates (Cellstar®, 655180) were seeded with 20,000 HepG2 cells in 200 μl media and left in an incubator (37° C., 5% CO₂) for 20 hours.

After incubation, the media was carefully aspirated from all of the wells and 200 μl of PRF media was added to the wells.

Pre-loaded doxorubicin beads and pre-loaded rapamycin beads were counted under aseptic conditions into 200 μl phenol red free cell culture media in a 96 well plate. The number of each beads placed in each well can be seen in table 5 (n=6). A control of each drug type was carried out alongside the assay (1-5 beads of one drug type only).

TABLE 6
Matrix showing beads ratios

| | Number of beads per well | | | | | | |
|---|---|---|---|---|---|---|---|
| Rapamycin beads | 1 | 1 | 1 | 1 | 2 | 3 | 4 |
| Doxorubicin beads | 1 | 2 | 3 | 4 | 1 | 1 | 1 |

After 72 hours exposure to the cells, an LDH assay as described in Example 6 was carried out on the controls and samples in Table 6.

An MTS assay was also conducted on the wells. At 72 hrs, the media from all wells was cautiously removed and each well washed three times with 200 μl sterile PBS. After adding 100 μl PRF media to each well, 20 μl of the prepared MTS solution (Promega) was also added. After a two hours incubation (37° C., 5% CO₂) the plate was read at 490 nm using the plate reader and like the LDH assay, if the reading was too high the samples were diluted (75 μl PRF media: 75 μl samples).

### Synergy of Rapamycin and Doxorubicin

The cell viability experiment with rapamycin beads and doxorubicin beads in different ratios allow any drug synergy to be established. A single rapamycin bead and a single doxorubicin bead together in one well left fewer viable cells than two beads of one drug type. This trend is seen throughout the bead combinations tested (FIGS. 15 & 16). A similar trend is noted with the MTS assay (FIGS. 17 & 18).

Using Calcusyn v2.1 (Biosoft) a combination index value can be established for each combination using the data from the LDH assay (Table 7). The value of the combination index relates to a scale (Table 7). Using these descriptions a strong synergism is noted under these conditions.

TABLE 6
Combination Index (CI) Values and description.

| Rapamycin (beads) | Doxorubicin (beads) | Fa | CI |
|---|---|---|---|
| 1 | 1 | 0.984 | 0.007 |
| 1 | 2 | 0.991 | 0.002 |
| 1 | 3 | 0.998 | 1.06e−5 |
| 1 | 4 | 0.994 | 0.001 |
| 2 | 1 | 0.997 | 1.52e−0.005 |
| 3 | 1 | 0.988 | 0.002 |

(Fa = fraction affected by the dose, 1 = 100% kill, 0 = no kill).

TABLE 7
Combination Index Values and description

| Range of CI | Description |
|---|---|
| <0.1 | +++++ Very strong synergism |
| 0.1-0.3 | ++++ Strong synergism |
| 0.3-0.7 | +++ Synergism |
| 0.7-0.85 | ++ Moderate synergism |
| 0.85-0.90 | + Slight synergism |
| 0.90-1.10 | ± Nearly additive |
| 1.10-1.20 | − Slight antagonism |
| 1.20-1.45 | −− Moderate antagonism |

As can be seen all doxorubicin-rapamycin combinations gave a CI that indicated a very strong synergism between the two drugs. Both assays show synergy between both drugs. The data do not show a preferred ratio, but synergy is shown for all combinations.

### EXAMPLE 8

### In Vitro Cytotoxicity of Rapamycin and Doxorubicin Loaded Beads Individually and in Combination

Example 7 was repeated substantially as described, with an extended number of beads of one of the drugs. The results of the MTS assay, i.e. showing cell viability % (log scale) of HepG2 cells after 72 hr exposure, concluded as the mean of 6 samples, is shown in FIG. 19.

### Results

When eluted individually, doxorubicin was more cytotoxic to the cells than rapamycin (FIG. 1). However, when used in combination, a further reduction in cell viability is observed. The combination indices for all bead combinations are <0.1; this indicates strong positive synergy between the two eluted drugs.

### EXAMPLE 9

### Synergy of Rapamycin and Doxorubicin on Human Pancreatic Carcinoma (PSN1) and Human Hepatocellular Carcinoma (HepG2) Cell Lines in-Vitro

An MTS assay was used to assess the synergistic cytotoxic effects of dual loaded beads on HepG2 and PSN1 cells. The rapamycin loaded beads and the doxorubicin loaded beads were the same as those described in Example 7. The dual loaded doxorubicin and rapamycin loaded beads were loaded and analysed as described in Example 4 Method 2 using a 1 ml 60 mg/ml rapamycin solution and 1 ml 37.5 mg/ml doxorubicin solution. The resulting loading in the dual loaded beads was 21.4 mg/ml rapamycin and 35.5 mg/ml doxorubicin.

### Assay Preparation

The assay was prepared as described in Example 7 using HepG2 cells and additional plates of PSN1 cells for analysis.

### Cell Viability

Pre-loaded doxorubicin beads, pre-loaded rapamycin beads and dual rapamycin and doxorubicin pre-loaded beads were counted (1, 2 or 3) and added to the prepared 20,000 PSN1 or HepG2 cells in cell culture media and placed in an incubator (37° C., 5% CO₂).

An MTS assay (described in example 7) was conducted after 24, 48 and 72 hrs exposure.

The dual loaded bead combination showed greater cell death than either mono-loaded bead at all time points for PSN1 cells (FIGS. 20-22) and HepG2 cells (FIG. 23-25).

### EXAMPLE 10

### Dual Loaded Bead Compared to Two Mono-Loaded Beads

An MTS assay was used to demonstrate that there was no difference in the cytotoxic effects of one doxorubicin and rapamycin dual-loaded bead compared to two mono-loaded beads (one doxorubicin loaded and one rapamycin loaded).

The drug loaded beads used were the same batches as those described in Example 9.

The assay was setup as described in Example 7 using HepG2 cells. One to four dual loaded beads were added to the prepared 20,000 HepG2 cells in 200 μl media and the equivalent dose using mono-loaded beads were added to another well in the same volume of media. The plates were placed in an incubator (37° C., 5% CO₂).

An MTS assay (described in example 7) was conducted after 24, 48 and 72 hrs exposure.

The cytotoxic effect of the drug eluting beads was similar regardless of whether the drugs were loaded in one bead or two separate beads (FIG. 26-28).

1. A composition comprising a population of microspheres each of which has a polymer matrix and, jointly incorporated into the matrix, a first and a second pharmaceutically active compound, wherein the polymer is a water-insoluble water-swellable polymer which is anionically charged, the first active is cationically charged, one of the actives is a cytotoxic compound and the other active has activity complementary to the cytotoxic compound in tumour treatment.

2. A composition according to claim 1, wherein the first cationically charged compound is a cytotoxic agent.

3. A composition according to claim 1, in which the second active is cationically charged.

4. A composition according to claim 1, in which the second active is electrostatically neutral.

5. A composition according to claim 4, in which the second active is water-soluble to a degree of less than 10 g/l at room temperature.

6. A composition according to claim 1, in which the two actives have a Combination Index of less than 0.9.

7. A pharmaceutical product comprises a first population of microspheres comprising a first polymer matrix and a first therapeutically active agent and a second population of microspheres comprising a second polymer matrix and a second therapeutically active agent, wherein said first and/or second polymer matrix(es) is (are) crosslinked water-swellable polymer and the first therapeutically agent and second therapeutically active agent are incorporated into the microspheres and the populations are present in a unit dose of product in amounts such that the Combination Index is less than 0.1.

8. A pharmaceutical composition according to claim 7, in which the drugs are jointly loaded into the same microspheres.

9. A pharmaceutical composition according to claim 7, in which the first active is a cytotoxic agent.

10. A composition according to claim 7, in which the polymer matrix is anionically charged and in which one of the actives is cationically charged.

11. A composition according to claim 1, in which the cytotoxic agent is mitoxantrone an anthracycline compound having an amine group, of the general formula IV

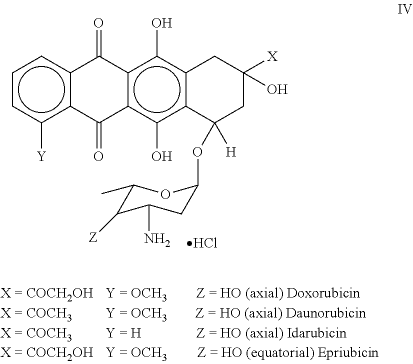

12. A composition according to claim 11, in which the anthracycline is doxorubicin.

13. A composition according to any claim 1, in which the cationic active is a compound of general formula I 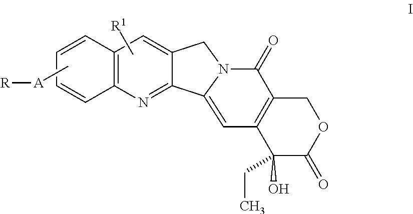
in which R¹ is selected from H, halogen, hydroxyl and lower (C₁₋₆) alkyl, optionally substituted by a hydroxyl, amine, alkoxy, halogen, acyl and acyloxy groups;
A is C(O)O or CH₂; and
R is NR²R³ where R² and R³ are the same or different and each represents a hydrogen atom, a substituted or unsubstituted C₁₋₄ alkyl group or a substituted or unsubstituted carbocyclic or heterocyclic group, or R² and R³ together with the nitrogen atom to which they are attached from a optionally substituted heterocyclic ring which may be interrupted by —O—, —S— or >NR⁴ in which R⁴ is a hydrogen atom, a substituted or unsubstituted C₁₋₄ alkyl group or a substituted or unsubstituted phenyl group;
and wherein the grouping -A-R is bonded to a carbon atom located in any of the 9, 10 or 11 positions in the A ring of the camptothecin compound and R¹ is substituted in the A or B ring, including salts thereof.

14. A composition according to claim 13, in which the cationic active is irinotecan.

15. A pharmaceutical composition according to claim 7, in which the second active agent is a compound having a water solubility at room temperature less than 10 g/l.

16. A compound according to claim 15, in which the low water-solubility compound is rapamycin or a rapamycin analogue.

17. A pharmaceutical composition according to claim 1, in which the second active agent is a COX inhibitor.

18. A pharmaceutical composition comprising a first population of microspheres comprising a first polymer matrix and a first therapeutic active, and a second population of microspheres comprising a second polymer matrix and a second therapeutically active compound, wherein said first and second polymer matrices are crosslinked water-insoluble water-swellable polymer, the first active is an anthracycline having an amine group substituent and the second active is rapamycin or an analogue.

19. A pharmaceutical composition according to claim 18, in which the first active is an anthracycline compound having an amine group, of the general formula IV

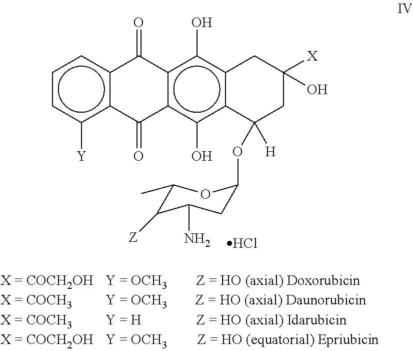

20. A pharmaceutical composition comprising a first population of microspheres comprising a first polymer matrix and a first therapeutic active, and a second population of microspheres comprising a second polymer matrix and a second therapeutically active compound, wherein said first and second polymer matrices are crosslinked water-insoluble water-swellable polymer, the first active is a compound of general formula I 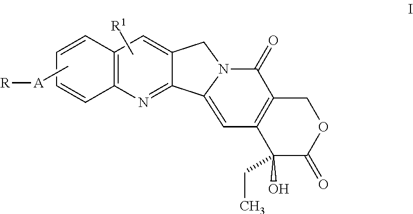
in which R¹ is selected from H, halogen, hydroxyl and lower (C₁₋₆) alkyl, optionally substituted by a hydroxyl, amine, alkoxy, halogen, acyl and acyloxy groups;
A is C(O)O or CH₂; and
R is NR²R³ where R² and R³ are the same or different and each represents a hydrogen atom, a substituted or unsubstituted C₁₋₄ alkyl group or a substituted or unsubstituted carbocyclic or heterocyclic group, or R² and R³ together with the nitrogen atom to which they are attached from a optionally substituted heterocyclic ring which may be interrupted by —O—, —S— or >NR⁴ in which R⁴ is a hydrogen atom, a substituted or unsubstituted C₁₋₄ alkyl group or a substituted or unsubstituted phenyl group;
and wherein the grouping -A-R is bonded to a carbon atom located in any of the 9, 10 or 11 positions in the A ring of the camptothecin compound and R¹ is substituted in the A or B ring, including salts thereof.

21. A pharmaceutical composition according to claim 18, in which the second active is rapamycin or a rapamycin analogue.

22. A pharmaceutical composition comprising a first population of microspheres comprising a first polymer matrix and a first therapeutic active, and a second population of microspheres comprising a second polymer matrix and a second therapeutically active compound, wherein said first and second polymer matrices are crosslinked water-insoluble water-swellable polymer, the first active is a compound of general formula I, 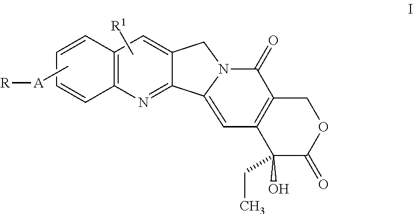
in which R¹ is selected from H, halogen, hydroxyl and lower (C₁₋₆) alkyl, optionally substituted by a hydroxyl, amine, alkoxy, halogen, acyl and acyloxy groups;
A is C(O)O or CH₂; and
R is NR²R³ where R² and R³ are the same or different and each represents a hydrogen atom, a substituted or unsubstituted C₁₋₄ alkyl group or a substituted or unsubstituted carbocyclic or heterocyclic group, or R² and R³ together with the nitrogen atom to which they are attached from a optionally substituted heterocyclic ring which may be interrupted by —O—, —S— or >NR⁴ in which R⁴ is a hydrogen atom, a substituted or unsubstituted C₁₋₄ alkyl group or a substituted or unsubstituted phenyl group;
and wherein the grouping -A-R is bonded to a carbon atom located in any of the 9, 10 or 11 positions in the A ring of the camptothecin compound and R¹ is substituted in the A or B ring, including salts thereof;
or is an anthracycline having an amine group substituent; and
the second compound is a COX inhibitor.

23. A pharmaceutical compound according to claim 20, in which the compound of general formula I is irinotecan.

24. A pharmaceutical composition according to claim 22, in which the COX inhibitor is ibuprofen.

25. A composition according to claim 1, in which the or each polymer is non-biodegradable.

26. A composition according to claim 1, in which the or each polymer is water-swellable to an equilibrium water content when swollen in water at 37° C., in the range 40 to 99%, preferably 75 to 95%.

27. A composition according to claim 1, in which the or each polymer is wholly synthetic.

28. A composition according to claim 27, in which the or each polymer is crosslinked polyvinyl alcohol.

29. A composition according to claim 28, wherein the polymer matrix is formed from a polyvinyl alcohol macromer, having more than one ethylenically unsaturated pendant group per molecule, by radical polymerisation of the ethylenic groups.

30. A composition according to claim 29, the macromer is copolymerised with ethylenically unsaturated monomers for instance including a nonionic and/or ionic monomer including anionic monomer.

31. A composition according to claim 30, where the ethylenically unsaturated monomers include an ionic monomer having the general formula III
Y¹BQ¹ III
in which Y¹ is selected from 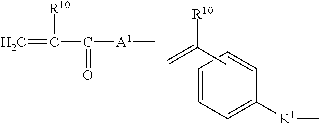
CH₂═C(R¹⁰)—CH₂—O—, CH₂═C(R¹⁰)—CH₂OC(O)—, CH₂═C(R¹⁰)OC(O)—, CH₂═C(R¹⁰)—O—, CH₂═C(R¹⁰)CH₂OC(O)N(R¹¹)—, R¹²OOCCR¹⁰═CR¹⁰C(O)—O—, R¹⁰CH═CHC(O)O—, R¹⁰CH═C(COOR¹²)CH₂—C(O)—O—, 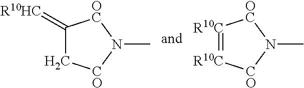
wherein:
R¹⁰ is hydrogen or a C₁-C₄ alkyl group;
R¹¹ is hydrogen or a C₁-C₄ alkyl group;
R¹² is hydrogen or a C₁₋₄ alkyl group or BQ¹ where B and Q¹ are as defined below;
A¹ is —O— or —NR¹¹—;
K¹ is a group —(CH₂)ᵣOC(O)—, —(CH₂)ᵣC(O)O—, —(CH₂)ᵣOC(O)O—, —(CH₂)ᵣNR¹³—, —(CH₂)ᵣNR¹³C(O)—, —(CH₂)ᵣC(O)NR¹³—, —(CH₂)ᵣNR¹³C(O)O—, —(CH₂)ᵣOC(O)NR¹³—, —(CH₂)ᵣNR¹³C(O)NR¹³— (in which the groups R¹³ are the same or different), —(CH₂)ᵣO—, —(CH₂)ᵣSO₃—, or, optionally in combination with B, a valence bond and r is from 1 to 12 and R¹³ is hydrogen or a C₁-C₄ alkyl group;
B is a straight or branched alkanediyl, oxaalkylene, alkanediyloxaalkanediyl, or alkanediyloligo(oxaalkanediyl) chain optionally containing one or more fluorine atoms up to and including perfluorinated chains or, if Q¹ or Y¹ contains a terminal carbon atom bonded to B a valence bond; and
Q¹ is an ionic group.

32. A composition according to claim 31, in which Q¹ is an anionic group, preferably a carboxylate, carbonate, sulphonate, sulphate, nitrate, phosphonate or phosphate group.

33. A composition according to claim 31, in which Q¹ is a cationic group, preferably a group N⁺R¹⁴₃, P⁺R¹⁵₃ or S⁺R¹⁵₂ in which the groups R¹⁴ are the same or different and are each hydrogen, C₁₋₄-alkyl or aryl (preferably phenyl) or two of the groups R¹⁴ together with the heteroatoms to which they are attached from a saturated or unsaturated heterocyclic ring containing from 5 to 7 atoms the groups R¹⁵ are each OR¹⁴ or R¹⁴.

34. A method of making a composition according to claim 3, in which aqueous solutions of each of the actives is, sequentially or simultaneously, contacted with the microspheres of polymer matrix whereby the two actives ion exchange to become electrostatically bound in the polymer matrix.

35. A method of loading two actives jointly into microspheres involving the steps:
contacting microspheres which comprise a matrix of a water-insoluble water-swellable anionically charged crosslinked polymer in substantially dry form with a solution of a drug having a water-solubility less than 10 g/l at room temperature in a first organic solvent, whereby a solution of drug in solvent becomes impregnated into the microspheres;
separating drug solution which has not impregnated the microspheres;
contacting the impregnated microspheres with aqueous liquid whereby drug is precipitated in the core of the microspheres;
contacting the microspheres containing precipitated drug with an aqueous solution of an ionic drug having a charge opposite to that of the polymer to allow loading of ion-exchange; and
recovering the dual loaded microspheres, wherein the drugs are selected as in claim 1.

36. A method of loading two pharmaceutical actives jointly into a drug delivery matrix comprising the steps:
a drug delivery matrix which comprises a water-insoluble, water-swellable ionically charged polymer which is swellable in water at room temperature to an equilibrium water content in the range 40 to 99% by weight based on polymer plus water is contacted with a solution of drug having a water-solubility less than 10 g/l at room temperature in an organic solvent which is capable of swelling the polymer, whereby the solution becomes impregnated into the matrix;
any drug solution which has not impregnated the polymer matrix is separated;
the impregnated matrix is contacted with water whereby drug is precipitated in the matrix;
the matrix having precipitated drug having low water solubility is contacted with an aqueous solution of an ionically charged drug whereby the matrix swells and ion-exchange with counterions associated with the polymer takes place and charged drug is electrostatically bound in the polymer matrix.

37. A method according to claim 35, in which the active having a water-solubility of less than 10 g/l is rapamycin or an analogue.

38. A method of loading two pharmaceutical actives jointly into microspheres comprising a matrix of water-insoluble water-swellable crosslinked ionically charged polymer, in which a first active is electrostatically charged with a charge opposite that of the polymer and the second active is a crystallisable drug,
in which the matrix is contacted with a solution of the crystallisable drug in a volatile organic solvent capable of swelling the matrix further containing a crystal modifier capable of inhibiting crystallisation of the drug,
the matrix is dried to remove the volatile solvent,
the matrix loaded with crystallisable drug and crystal modifier is contacted with an aqueous solution of the ionic drug whereby the matrix swells and ion-exchange with counterions associated with the polymer takes place and drug is electrostatically bound in the polymer matrix.

39. A method according to claim 38, in which the second drug is a COX inhibitor.

40. A method according to claim 38, in which the crystal modifier is a crystallisation inhibitor.

41. A method according to claim 36, in which the ionically charged active is a compound of general formula I 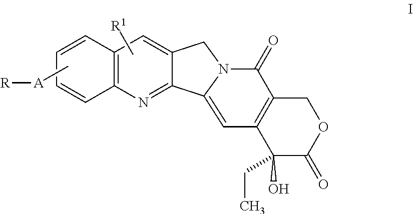
in which R¹ is selected from H, halogen, hydroxyl and lower (C₁₋₆) alkyl, optionally substituted by a hydroxyl, amine, alkoxy, halogen, acyl and acyloxy groups;
A is C(O)O or CH₂, and
R is NR²R³ where R² and R³ are the same or different and each represents a hydrogen atom, a substituted or unsubstituted C₁₋₄ alkyl group or a substituted or unsubstituted carbocyclic or heterocyclic group, or R² and R³ together with the nitrogen atom to which they are attached from a optionally substituted heterocyclic ring which may be interrupted by —O—, —S— or >NR⁴ in which R⁴ is a hydrogen atom, a substituted or unsubstituted C₁₋₄ alkyl group or a substituted or unsubstituted phenyl group;
and wherein the grouping -A-R is bonded to a carbon atom located in any of the 9, 10 or 11 positions in the A ring of the camptothecin compound and R¹ is substituted in the A or B ring, including salts thereof.

42. A method according to claim 41, in which the compound of general formula I is irinotecan.

43. A method according to claim 36, in which the ionically charged active is a mitoxantrone or anthracycline compound having an amine group.

44. A method according to claim 43, in which the anthracycline is an anthracycline compound having an amine group, of the general formula IV

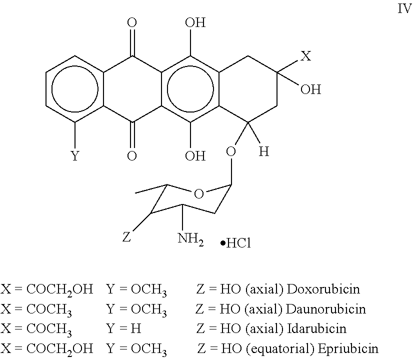

45. A method according to claim 34, in which the two actives have a Combination Index of less than 0.9, preferably less than 0.1.

46. A method according to claim 34, in which the polymer is a composition comprising a population of microspheres each of which has a polymer matrix and, jointly incorporated into the matrix, a first and a second pharmaceutically active compound, wherein the polymer is a water-insoluble water-swellable polymer which is anionically charged, the first active is cationically charged, one of the actives is a cytotoxic compound and the other active has activity complementary to the cytotoxic compound in tumour treatment and in which the or each polymer is wholly synthetic.

47. Method of treating a solid tumour by administering a composition according to claim 1 to an animal suffering from a tumour in such a manner that the first and second actives are each delivered at the target tumour.

48. Method according to claim 47, in which the composition is administered by being injected intratumourally.

49. Method according to claim 47, in which the composition is injected at the resection margin after removal of the tumour by surgery.

50. Method according to claim 47, in which the matrix is in the form of microspheres, having diameters in the range 100 to 1500 μm when swollen in distilled water at 37° C., by embolisation of a blood vessel feeding the tumour.

51. Method according to claim 46, in which the tumour is a hepatic tumour.

52. A composition according to claim 6, in which the two actives have a Combination Index of less than 0.1.

53. A compound according to claim 16, in which the low water-solubility compound is rapamycin.

54. A pharmaceutical composition according to claim 17, in which the second active agent is ibuprofen.

55. A pharmaceutical composition according to claim 19, in which the first active is doxorubicin.

56. A method according to claim 39, in which the second drug is ibuprofen.

57. A method according to claim 40, in which the crystal modifier is an oil.
